(19) United States
(12) Patent Application Publication
(10) Pub. No.: US 2023/0326552 A1
(43) Pub. Date: Oct. 12, 2023

(54) SYSTEMS AND METHODS FOR COMPUTATIONAL DESIGN OF CRISPR GUIDE RNAS FOR STRAIN-SPECIFIC CONTROL OF MICROBIOTA CONSORTIA

(71) Applicant: Washington University, St. Louis, MO (US)

(72) Inventors: Tae Seok Moon, St. Louis, MO (US); Austin Rottinghaus, St. Louis, MO (US)

(73) Assignee: Washington University, St. Louis, MO (US)

(21) Appl. No.: 18/297,497

(22) Filed: Apr. 7, 2023

Related U.S. Application Data
(60) Provisional application No. 63/328,628, filed on Apr. 7, 2022.

(51) Int. Cl. G16B 20/50; C12N 9/22; C12N 15/10

(52) U.S. Cl. CPC G16B 20/50; C12N 9/22; C12N 15/102; C12N 2310/20

(57) ABSTRACT

Among the various aspects of the present disclosure is the provision of systems and methods for producing systems and methods for the computational design of CRISPR guide RNAs for strain-specific control of microbiota consortia.

### CROSS-REFERENCE TO RELATED APPLICATIONS

This application claims priority from U.S. Provisional Application Ser. No. 63/328,628 filed on Apr. 7, 2022, which is incorporated herein by reference in its entirety.

### STATEMENT REGARDING FEDERALLY SPONSORED RESEARCH OR DEVELOPMENT

This invention was made with government support under R840205-01 awarded by the Environmental Protection Agency (EPA), AT009741 awarded by the National Institutes of Health (NIH), CBET-1350498 and MCB-1714352 awarded by the National Science Foundation (NSF), N00014-17-1-2611 and N00014-19-1-2357 awarded by the Office of Naval Research (ONR), and 2020-33522-32319 awarded by the United States Department of Agriculture (USDA). The government has certain rights in the invention.

### MATERIAL INCORPORATED-BY-REFERENCE

The Sequence Listing, which is a part of the present disclosure, includes a computer-readable form comprising nucleotide and/or amino acid sequences of the present invention entitled ‘020152-US-NP_SEQUENCE_LISTING.XML’, created on Apr. 6, 2023, and sized at 103,222 bytes. The subject matter of the Sequence Listing is incorporated herein by reference in its entirety.

### FIELD OF THE DISCLOSURE

The present disclosure generally relates to systems and methods for the computational design of CRISPR guide RNAs. In particular, the present disclosure generally relates to systems and methods for the computational design of CRISPR guide RNAs for strain-specific control of microbiota consortia.

### BACKGROUND OF THE DISCLOSURE

Microbes naturally co-exist in complex and dynamic communities. These microbial consortia cooperate to influence the health of the environment, domestic animals, humans, and plants.

Efforts to create synthetic microbial communities have led to advances in fields including metabolic engineering and bioremediation. Numerous microbes have been extracted from natural consortia with highly specialized and essential functions. However, identifying and purifying these microbes remains challenging. Pathogens also inhabit these communities and opportunistically disrupt host health. Modern methods of removing them, including antibiotics, are highly disruptive to the survival of homeostatic, beneficial microbes and have led to the global emergence of deadly antibiotic- and bactericide-resistant pathogens. Recent advances in phage engineering and plasmid conjugation have allowed microbes to be targeted and killed in a strain-specific manner causing minimal impact on the stability of the microenvironment. Microbes have also been engineered with novel functions and introduced into natural microbiomes to improve the health of the host and engineered to selectively colonize specific microenvironments. However, exogenously provided microbes often have a difficult time penetrating consortia, finding a niche, and persisting long-term. As an alternative to supplementing microbiota with engineered microbes, microbes can instead be engineered in situ using external DNA delivery methods, increasing the endurance of the added functionality. However, methods for engineering microbes in situ often lack strain specificity and instead introduce the exogenous DNA randomly into the microbiota.

CRISPR-Cas systems can be tuned to recognize specific genetic loci by modulating the sequence of the guide RNA (gRNA), providing opportunities for strain recognition in microbial consortia. This functionality has been harnessed for applications in strain-specific microbial engineering and elimination. Numerous programs have been developed to help design gRNAs with high cutting efficiency and low off-target cleavage rates using machine learning and deep learning models that consider the sequence and thermodynamic characteristics of the gRNA sequence. However, programs for designing gRNAs specific to individual microbial strains are lacking. One recent work achieved this goal with an effective and accessible website. However, the program lacks strain selection options, cannot be utilized for diverse CRISPR systems beyond Cas9, and defines a strain-specific gRNA as one with at least one nucleotide (nt) mismatch in the non-target strains, which has been shown to be insufficient to prevent cleavage.

Other objects and features will be in part apparent and in part pointed out hereinafter.

### SUMMARY OF THE INVENTION

Among the various aspects of the present disclosure is the provision of systems and methods for a computer-implemented design of CRISPR guide RNAs for strain-specific control of microbiota consortia.

Briefly, therefore, the present disclosure is directed to strain-specific control of microbiota consortia with systems and methods for computational design of CRISPR guide RNAs.

In one aspect, a computer-implemented method of producing at least one gRNA sequence for use in CRISPR-Cas gene editing of microbial organisms is disclosed. Each gRNA sequence includes a protospacer adjacent motif (PAM) sequence and a target nucleotide sequence. The method includes receiving, at a computing device, at least one non-target strain genome sequence, at least one target strain genome sequence, the PAM sequence, a PAM orientation, a specificity threshold, and a target length, identifying, using the computing device, a plurality of candidate gRNA sequences within the at least one target strain genome sequence, based on the PAM nucleotide sequence, the PAM orientation, and the target length. The method also includes selecting, using the computing device, at least one broad-specificity gRNA sequence from the plurality of candidate gRNA sequences, wherein each broad-specificity gRNA sequence is contained within all of the at least one target strain genome sequences. The method also includes identifying, using the computing device, a plurality of non-target gRNA sequences within the at least one non-target strain genome sequence, based on the PAM nucleotide sequence, the PAM orientation, and the target length, and selecting, using the computing device, at least one strain-specific gRNA sequence from the at least one broad-specificity gRNA sequence based on the specificity threshold, wherein the at least one strain-specific gRNA sequence is not contained within any of the non-target strain gRNA sequences. In some aspects, the candidate gRNA sequence, broad-specificity gRNA sequence, non-target gRNA sequence, non-target gRNA sequence, and strain-specific gRNA sequence each comprise the PAM nucleotide sequence and a target nucleotide sequence comprising the target length of nucleotides, the PAM nucleotide sequence and the target sequence arranged according to the PAM orientation selected from 5′-(PAM nucleotide sequence)-(target nucleotide sequence)-3′ or 5′-(target nucleotide sequence)-PAM nucleotide sequence)-3′. In some aspects the specificity threshold comprises a minimum number of nucleotide mismatches between the target sequence of each strain-specific gRNA sequence and the target sequence of all non-target strain gRNA sequences. In some aspects, the specificity threshold ranges from 0 nucleotides (nt) to about 4 nt. In some aspects, the specificity threshold is at least 3 nt. In some aspects, the target length ranges from about 10 nt to about 20 nt. In some aspects, the target length is 20 nt. In some aspects, selecting the at least one strain-specific gRNA sequence from the at least one broad-specificity gRNA sequence further includes generating, using the computing device, nucleotide sequence permutations within each target region of each non-target gRNAS sequence ranging from at least one nucleotide up to the specificity threshold number of nucleotides relative to the at least one non-target gRNA sequence, and comparing, using the computing device, each target sequence of each broad-specificity gRNA sequence to the plurality of nucleotide sequence permutations and discarding each broad-specificity gRNA sequence that matches any of the nucleotide sequence permutations, and selecting the remaining broad-specificity gRNA sequences as the strain-specific gRNA sequences.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 is a block diagram schematically illustrating a system in accordance with one aspect of the disclosure.

FIG. 2 is a block diagram schematically illustrating a computing device in accordance with one aspect of the disclosure.

FIG. 3 is a block diagram schematically illustrating a remote or user computing device in accordance with one aspect of the disclosure.

FIG. 4 is a block diagram schematically illustrating a server system in accordance with one aspect of the disclosure.

FIG. 5 is a flowchart showing the ssCRISPR program logic for strain-specific gRNA design. The user first inputs the desired non-target strains, target strains, nucleotides of specificity (1-4 nt), PAM sequences and orientation (5′ or 3′), and target length (grey). The program searches the first selected target strain for all potential gRNA target sites using the user-specified PAM sequence, PAM orientation, and target length. Next, the program iterates through all additional selected target strains and identifies the gRNA target sequences that are perfectly shared between the strains (green). The program then identifies gRNA target sites in batches of non-target strains and eliminates gRNAs that have less than the threshold number of nucleotides defining specificity for any non-target strain (blue). Finally, for Cas9 and Cpf1 gRNAs, the program predicts the relative efficiencies of the determined gRNAs using 396 sequence composition and energetic properties. The gRNAs are ranked by their relative efficiency and a full report of the results is provided to the user (yellow). The number of gRNAs tested for specificity is capped at *10,000 for 2 nt mismatches and **100 for 3 nt mismatches due to limits in computation power.

FIG. 6A is a graph showing the number of gRNAs that broadly target different amounts of each of the 2,068 E. coli and 1,020 Pseudomonas strains.

FIG. 6B is a heat map plot of the actual versus predicted efficiency rankings for 56,335 Cas9 gRNAs.

FIG. 6C is a graph of the efficiency values for the top four predicted gRNAs that target all E. coli strains in E. coli DH10B, Nissle 1917, MG1655, and BL21(DE3). Efficiency values were obtained using cell death transformation assays. Efficiency values are the ratio of the number of colonies obtained from each gRNA plasmid to the number of colonies obtained from the control plasmid with Cas9 present divided by the ratio of colonies obtained from each gRNA plasmid to the number of colonies obtained from the control plasmid without Cas9 present (see Methods of). Values and error bars are the average and standard deviation of biological triplicate, respectively. Source data are provided as a source data file.

FIG. 6D is a graph of the efficiency values for the top four predicted gRNAs that target all Pseudomonas strains in P. putida F1, P. putida KT2440, P. stutzeri JM300, and P. syringae pv. tomato DC3000. Efficiency values were obtained using cell death transformation assays. Efficiency values are the ratio of the number of colonies obtained from each gRNA plasmid to the number of colonies obtained from the control plasmid with Cas9 present divided by the ratio of colonies obtained from each gRNA plasmid to the number of colonies obtained from the control plasmid without Cas9 present (see Methods). Values and error bars are the average and standard deviation of biological triplicate, respectively. Source data are provided as a source data file.

FIG. 7A is a heat map plot of the efficiency of the top-scoring strain-specific gRNAs with at least one mismatched nucleotide (nt) in the PAM or at least (left) 1 mismatched nucleotide in the 10 nt PAM-adjacent target region. gRNAs were designed to selectively target E. coli DH10B, Nissle 1917, MG1655, or BL21(DE3). The top four predicted gRNAs for each strain were selected from the program and tested for killing efficiency using a transformation assay. Efficiency values are the ratio of the number of colonies obtained from each gRNA plasmid to the number of colonies obtained from the control plasmid with Cas9 present divided by the ratio of colonies obtained from each gRNA plasmid to the number of colonies obtained from the control plasmid without Cas9 present. Each value is the average of biological duplicates. Source data are provided as a source data file.

FIG. 7B is a heat map plot of the efficiency of the top-scoring strain-specific gRNAs with at least one mismatched nucleotide (nt) in the PAM or at least 2 mismatched nucleotides in the 10 nt PAM-adjacent target region. gRNAs were designed to selectively target E. coli DH10B, Nissle 1917, MG1655, or BL21(DE3). The top four predicted gRNAs for each strain were selected from the program and tested for killing efficiency using a transformation assay. Efficiency values are the ratio of the number of colonies obtained from each gRNA plasmid to the number of colonies obtained from the control plasmid with Cas9 present divided by the ratio of colonies obtained from each gRNA plasmid to the number of colonies obtained from the control plasmid without Cas9 present. Each value is the average of biological duplicates. Source data are provided as a source data file.

FIG. 7C is a heat map plot of the efficiency of the top-scoring strain-specific gRNAs with at least one mismatched nucleotide (nt) in the PAM or at least 3 mismatched nucleotides in the 12 nt PAM-adjacent target region. gRNAs were designed to selectively target E. coli DH10B, Nissle 1917, MG1655, or BL21(DE3). The top four predicted gRNAs for each strain were selected from the program and tested for killing efficiency using a transformation assay. Efficiency values are the ratio of the number of colonies obtained from each gRNA plasmid to the number of colonies obtained from the control plasmid with Cas9 present divided by the ratio of colonies obtained from each gRNA plasmid to the number of colonies obtained from the control plasmid without Cas9 present (see methods). Each value is the average of biological duplicates. Source data are provided as a source data file.

FIG. 7D is a heat map plot of the efficiency of strain-specific gRNAs with at least one mismatched nucleotide in the PAM or at least 3 mismatched nucleotides in the 20 nt PAM-adjacent target region. gRNAs were designed to selectively target E. coli DH10B, Nissle 1917, MG1655, or BL21(DE3). The top four predicted gRNAs for each strain were selected from the program and tested for killing efficiency using a transformation assay. Efficiency values are the ratio of the number of colonies obtained from each gRNA plasmid to the number of colonies obtained from the control plasmid with Cas9 present divided by the ratio of colonies obtained from each gRNA plasmid to the number of colonies obtained from the control plasmid without Cas9 present (see methods). Each value is the average of biological or triplicate. Source data are provided as a source data file.

FIG. 7E is a heat map plot of the efficiency of strain-specific gRNAs with at least one mismatched nucleotide in the PAM or at least 3 mismatched nucleotides in the 20 nt PAM-adjacent target region. gRNAs were designed to selectively target P. putida F1, P. putida KT2440, P. stutzeri JM3000, or P. syringae pv. tomato DC3000. The top four predicted gRNAs for each strain were selected from the program and tested for killing efficiency using a transformation assay. Efficiency values are the ratio of the number of colonies obtained from each gRNA plasmid to the number of colonies obtained from the control plasmid with Cas9 present divided by the ratio of colonies obtained from each gRNA plasmid to the number of colonies obtained from the control plasmid without Cas9 present. Each value is the average of biological triplicate. Source data are provided as a source data file.

FIG. 8A is a schematic diagram illustrating a procedure for isolating and engineering specific strains from a consortium. Selected consortia are transformed with a Cas9- and lambda red-containing CRISPR plasmid. The strain mixture with the CRISPR plasmid is then transformed with a strain-specific gRNA plasmid, designed to target selected non-desired strains, and double-stranded DNA carrying an antibiotic resistance gene (ARG). The ARG is integrated into the genome by lambda red recombinase to yield antibiotic-resistant microbes. Recombinants are then isolated by plating on agar plates containing the relevant antibiotic. The transformed gRNA plasmid selectively kills non-desired strains, leaving viable colonies only of the desired microbe. A second round of recombination can be performed using an ARG-specific gRNA to replace the ARG with any DNA of interest (DOI).

FIG. 8B is a graph of CFUs/Transformation from an isolation of E. coli Nissle 1917 from a three-microbe consortium with E. coli DH10B and MG1655. Cultures of each of the three microbes alone and together at a 1:1:1 ratio were transformed with a kanamycin-resistance cassette and either an empty control plasmid (gRNA-) or a plasmid harboring a gRNA designed to target E. coli DH10B and MG1655, but not E. coli Nissle 1917.

FIG. 8C is a plasmid schematic summarizing the plasmids used for strain-specific isolation of microbes from consortia. Six gRNAs were designed to target different subsets of the Enterobacteriaceae family while protecting E. coli Nissle 1917. Each gRNA is expressed in a unique cassette with nonrepetitive constitutive promoters, Cas9 hairpins, terminators, and spacer regions.

FIG. 8D is a graph of the fraction population from an isolation of E. coli Nissle from defined single-genus and multi-genus microbial consortia. Cultures of E. coli DH10B, MG1655, BL21(DE3), and Nissle 1917 or E. coli Nissle 1917, P. putida F1, S. typhimurium, and R. opacus PD630 were mixed at a 1:1:1:1 ratio and transformed with an empty control plasmid or a plasmid harboring a constitutive Cas9 cassette and an Enterobacteriaceae-targeting but E. coli Nissle-protecting gRNA array. Strains were quantified by next-generation amplicon sequencing (left) or qPCR (right). Values and error bars are the average and standard deviation of biological triplicate, respectively. Statistical comparisons between the control plasmid and gRNA array plasmid were performed using two-sided two-way ANOVA with Sidak's multiple comparisons (***, p<0.001; ****, p<0.0001).

FIG. 9A is a graph of the CFUs/Transformation showing that plasmids harboring constitutive Cas9 and E. coli Nissle-specific gRNAs selectively remove E. coli Nissle from microbial consortia. Defined consortia of a 1:1:1:1 mixture of E. coli DH10B, MG1655, BL21(DE3), and Nissle 1917 were transformed with a control plasmid or an E. coli Nissle-specific targeting plasmid and the strains identified by antibiotic plating. The fold difference in CFUs between transformation with the control plasmid and E. coli Nissle-specific plasmid was then quantified.

FIG. 9B is a graph of the CFUs/Transformation showing that plasmids harboring constitutive Cas9 and E. coli Nissle-specific gRNAs selectively remove E. coli Nissle from microbial consortia. Defined consortia of mouse fecal samples containing ˜2% E. coli Nissle were transformed with a control plasmid or an E. coli Nissle-specific targeting plasmid and the strains identified by antibiotic plating. The fold difference in CFUs between transformation with the control plasmid and E. coli Nissle-specific plasmid was then quantified.

FIG. 9C is a schematic illustration showing strain-specific antimicrobial liposomes. Cationic liposomes packaged with plasmids harboring Cas9 and strain-specific gRNA cassettes are delivered to complex microbial consortia. Liposomes nonspecifically fuse with microbes, delivering the payload. Microbes harboring the gRNA target sequence have their genome inactivated by Cas9 cleavage, causing cell death.

FIG. 9D is a graph of CFUs and fold difference of E. coli DH10B, MG1655, BL21(DE3), and Nissle 1917 that received control plasmid and E. coli Nissle-specific plasmid payloads after incubation with DNA-loaded liposomes. Values and error bars are the average and standard deviation of biological triplicate, respectively. Statistical comparisons between the control plasmid and the Nissle-specific plasmid were performed using two-sided one-way ANOVA with Tukey's Honest Significant Difference post-hox test (***, p<0.001; ****, p<0.0001).

FIG. 10 is a graph of the number of gRNAs identified by ssCRISPR that target all E. coli strains or all Pseudomonas strains with PAM sequences, target site lengths, and target-PAM orientations specific to variants of Cas9, Cas3, Cas12b, and Cpf1.

FIG. 11A is a nucleotide schematic for specified regions of the target sgRNA-DNA duplex. T20, the full 20 nucleotide duplex; T5, the five nucleotides immediately adjacent to the PAM site; T8, the eight nucleotides adjacent to T5; T7, the final seven nucleotides furthest from the PAM site.

FIG. 11B is a graph of the permutation importance for 12 thermodynamic and sequence features. Permutation importance is quantified as the reduction in model accuracy that occurs when the values of the respective feature are randomly shuffled. Tm, melting temperature; MFE, minimum free energy.

FIG. 11C is a graph of the presence of an A nucleotide in each position of the gRNA recognition sequence.

FIG. 11D is a graph of the presence of a T nucleotide in each position of the gRNA recognition sequence.

FIG. 11E is a graph of the presence of a G nucleotide in each position of the gRNA recognition sequence.

FIG. 11F is a graph of the presence of a C nucleotide in each position of the gRNA recognition sequence.

FIG. 12A is a heat map plot of the actual versus predicted efficiency rankings for 15,000 Cpf1 gRNAs. Predicted efficiency rankings were determined using a modified machine-learning approach. Machine learning models were trained using a gRNA efficiency dataset specific to Cas9.

FIG. 12B is a heat map plot of the actual versus predicted efficiency rankings for 15,000 Cpf1 gRNAs. Predicted efficiency rankings were determined using a modified machine-learning approach. Machine learning models were trained using a gRNA efficiency dataset specific to Cpf1.

FIG. 13 is a graph of the probability that a gRNA target site will occur in a randomly generated sequence of different lengths in million nucleotides (Mnt) when using different criteria for specificity: at least 1 nucleotide (nt) mismatch in the PAM or at least (grey) 1 nt in the 10 nt PAM-adjacent target region or 3 nt in the 12 nt PAM-adjacent target region, (light red) 2 nt in the 10 nt PAM-adjacent target region, (red) 3 nt in the 10 nt PAM-adjacent target region, and (dark red) 3 nt in the 20 nt PAM-adjacent target region.

FIG. 14A is a graph of computation time and number of strain-specific gRNAs with 3 nucleotides (nt) of specificity when the number of gRNAs with 2 nt of specificity is limited to different values. The gRNAs were designed to target E. coli Nissle 1917 and protect E. coli DH10B, MG1655, and BL21(DE3). The program generates a computer RAM-dependent memory error at a limit of about 31,000 gRNAs with 2 nt of specificity.

FIG. 14B is a graph of computation time and number of strain-specific gRNAs with 4 nt of specificity when the number of gRNAs with 3 nt of specificity is limited to different values. The gRNAs were designed to target E. coli Nissle 1917 and protect E. coli DH10B, MG1655, and BL21(DE3). The program generates a computer RAM-dependent memory error at a limit of about 470 gRNAs with 3 nt of specificity.

FIG. 15A is a graph of the number of gRNAs that target E. coli DH10B, Nissle 1917, MG1655, or BL21(DE3) while protecting the remaining three strains with 0, 1, 2, or 3 nucleotides (nt) of specificity.

FIG. 15B is a graph of the number of gRNAs that target P. putida F1, P. putida KT2440, P. stutzeri JM3000, or P. syringae pv. Tomato DC3000 while protecting the remaining three strains with 0, 1, 2, or 3 nucleotides of specificity.

FIG. 15C is a graph of the number of gRNAs that target E. coli DH10B, Nissle 1917, MG1655, or BL21(DE3) while protecting all other E. coli strains with 2 or 3 nucleotides of specificity.

FIG. 15D is a graph of the number of gRNAs that target P. putida F1, P. putida KT2440, P. stutzeri JM3000, or P. syringae pv. tomato DC3000 while protecting all other Pseudomonas strains with 2 or 3 nucleotides of specificity.

FIG. 16A is a graph of gRNA killing efficiency. Two gRNAs were designed to target diverse Enterobacteriaceae while protecting E. coli Nissle 1917 with one nucleotide of specificity. The gRNA killing efficiencies in E. coli Nissle 1917 were determined using a cell death transformation assay. One gRNA (red line) was selected for each strain group that best protected E. coli Nissle 1917. Values and error bars are the average and standard deviation of biological triplicate, respectively. Statistical comparisons to determine if the gRNA efficiencies were non-zero were performed using two-sided unpaired t-tests (*, p<0.05; **, p<0.01; ***, p<0.001).

FIG. 16B is a graph of the gRNA-killing efficiencies for the six selected gRNAs in E. coli MG1655. Efficiency values are the ratio of the number of colonies obtained from each gRNA plasmid to the number of colonies obtained from the control plasmid with Cas9 present divided by the ratio of colonies obtained from each gRNA plasmid to the number of colonies obtained from the control plasmid without Cas9 present. Values and error bars are the average and standard deviation of biological triplicate, respectively. Statistical comparisons to determine if the gRNA efficiencies were non-zero were performed using two-sided unpaired t-tests (*, p<0.05; **, p<0.01; ***, p<0.001).

FIG. 17A is a graph of the fraction of P. putida F1 from defined Pseudomonas consortia. Cultures of each of P. putida F1, P. putida KT2440, P. stutzeri JM300, and P. syringae pv. tomato DC3000 were mixed at a 1:1:1:1 ratio and transformed with an empty control plasmid or a plasmid harboring a constitutive Cas9 cassette and a Pseudomonas-targeting but P. putida F1-protecting gRNA. Strains were quantified by next-generation amplicon sequencing. Values and error bars are the average and standard deviation of biological triplicate, respectively.

FIG. 17B is a graph of the ratio of P. putida F1 to P. putida KT2440, P. stutzeri JM300, and P. syringae pv. tomato DC3000 obtained from the next-generation amplicon sequencing samples. Values and error bars are the average and standard deviation of biological triplicate, respectively. Statistical comparisons were performed using two-sided two-way ANOVA with Sidak's multiple comparisons (****, p<0.0001).

FIG. 18 is a pair of two graphs quantifying fecal bacteria titers from mice gavaged with E. coli Nissle 1917. Total bacterial and E. coli Nissle 1917-specific titers in murine fecal samples from mice gavaged with 10⁸ CFUs E. coli Nissle 1917. E. coli Nissle 1917 made up an average of 2% of the total culturable microbes in the fecal samples. Values and error bars are the average and standard deviation of biological triplicate, respectively.

FIG. 19 is a graph of the CFUs of E. coli Nissle 1917 that successfully received control plasmid DNA from DNA-loaded liposomes. Liposomes were sonicated for 5 and 30 min in a bath sonicator during formation. Liposomes were packaged with control plasmid DNA and delivered to bacterial cultures at the varying specified lipid concentrations. Values and error bars are the average and standard deviation of biological duplicates, respectively.

There are shown in the drawings arrangements that are presently discussed, it being understood, however, that the present embodiments are not limited to the precise arrangements and are instrumentalities shown. While multiple embodiments are disclosed, still other embodiments of the present disclosure will become apparent to those skilled in the art from the following detailed description, which shows and describes illustrative aspects of the disclosure. As will be realized, the invention is capable of modifications in various aspects, all without departing from the spirit and scope of the present disclosure. Accordingly, the drawings and detailed description are to be regarded as illustrative in nature and not restrictive.

### DETAILED DESCRIPTION

In various aspects, systems and methods for the computational design of CRISPR guide RNAs for strain-specific control of microbiota consortia are disclosed herein. In some aspects, the systems and methods make use of a program, ssCRISPR, that computationally designs strain-specific CRISPR gRNAs from user-defined target and non-target strains in various aspects. In some aspects, the systems and methods provide for the selection of target and non-target strain sequences from a database of genome sequences for strain options as extracted from the expansive National Center for Biotechnology Information (NCBI) genome repository, giving users over 27,000 strain selection options. In other aspects, the systems and methods may receive user-provided genome sequences in one implementation of the disclosed method. In some aspects, users of ssCRISPR can also input a desired protospacer adjacent motif (PAM) sequence, target sequence length, and PAM-target orientation, to render the disclosed systems and methods compatible with any CRISPR-Cas system.

In addition, users can select their desired criteria for specificity, from 1-4 nt, as the application will dictate the required stringency. However, as described in the Examples, at least 3 nt mismatches in the target sequence relative to the genomes of all non-target strains may assure to ensure complete strain-specificity. Herein, we demonstrated two potential applications of ssCRISPR-designed gRNAs: first, the purification of a single strain from a microbial consortium using a single plasmid transformation, and second, the in situ depletion of a single strain from a microbial consortium using liposomal delivery of strain-specific CRISPR-Cas9 cassettes. ssCRISPR can be downloaded and run locally either as a Python script or as an all-encompassing executable application. In either case, users can take advantage of the user-friendly graphical interface to operate the program without programming expertise.

In various aspects, at least a portion of the disclosed whole-genome sequencing methods may be implemented using various computing systems and devices as described below.

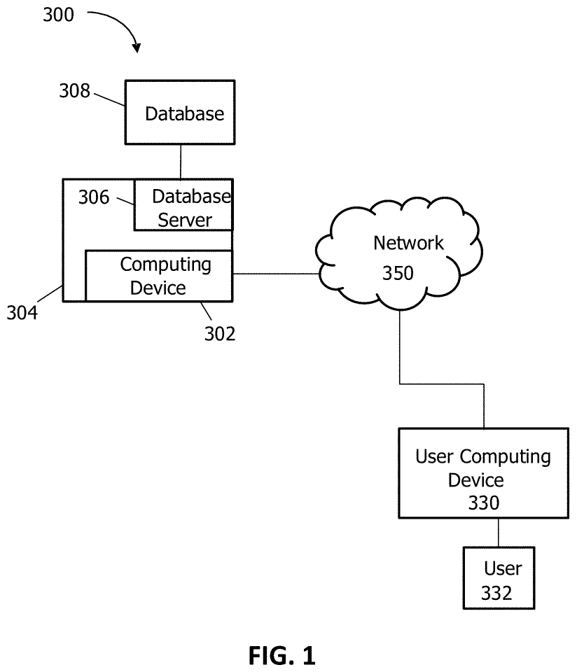

FIG. 1 depicts a simplified block diagram of a computing device for implementing the methods described herein. As illustrated in FIG. 1, the computing device 300 may be configured to implement at least a portion of the tasks associated with the disclosed method. The computer system 300 may include a computing device 302. In one aspect, the computing device 302 is part of a server system 304, which also includes a database server 306. The computing device 302 is in communication with a database 308 through the database server 306. The computing device 302 is communicably coupled to a user-computing device 330 through a network 350. The network 350 may be any network that allows local area or wide area communication between the devices. For example, the network 350 may allow communicative coupling to the Internet through at least one of many interfaces including, but not limited to, at least one of a network, such as the Internet, a local area network (LAN), a wide area network (WAN), an integrated services digital network (ISDN), a dial-up-connection, a digital subscriber line (DSL), a cellular phone connection, and a cable modem. The user-computing device 330 may be any device capable of accessing the Internet including, but not limited to, a desktop computer, a laptop computer, a personal digital assistant (PDA), a cellular phone, a smartphone, a tablet, a phablet, wearable electronics, smartwatch, or other web-based connectable equipment or mobile devices.

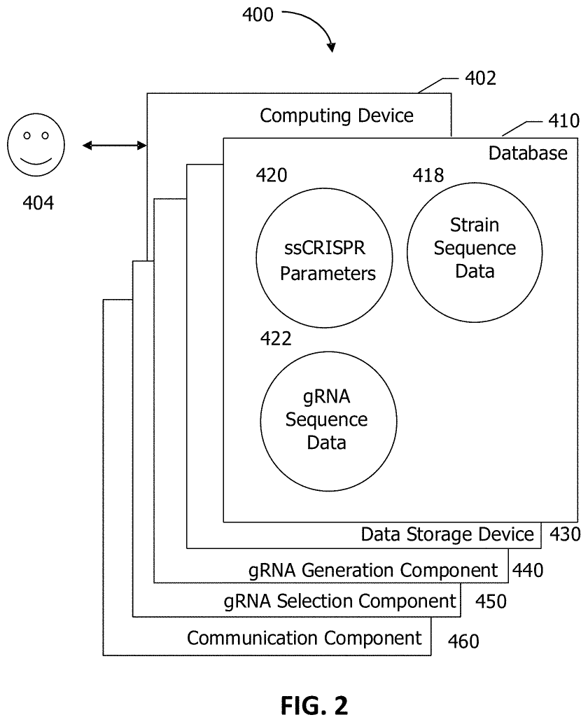

In other aspects, the computing device 302 is configured to perform a plurality of tasks associated with the disclosed method. FIG. 2 depicts a component configuration 400 of computing device 402, which includes database 410 along with other related computing components. In some aspects, computing device 402 is similar to computing device 302 (shown in FIG. 1). A user 404 may access components of computing device 402. In some aspects, database 410 is similar to database 308 (shown in FIG. 1).

In one aspect, database 410 includes strain sequence data 418, ssCRISPR parameters 420, and gRNA sequence data 422. Non-limiting examples of suitable strain sequence data 418 include whole-genome sequences of a plurality of bacterial strains, including, but not limited to target bacterial strains and non-target bacterial strains. In various aspects, the strain sequence data 418 may be pre-loaded from a whole-genome library or may be user-specified. Non-limiting examples of suitable ssCRISPR parameters 420 include any values of parameters defining the disclosed method including, but not limited to, PAM sequences and orientations, specificity thresholds, and target lengths. Non-limiting examples of suitable gRNA sequence data 422 include any gRNA sequences produced using the systems and methods disclosed herein.

Computing device 402 also includes a number of components that perform specific tasks. In the exemplary aspect, computing device 402 includes a data storage device 430, a gRNA generation component 440, a gRNA selection component 450, and a communication component 460. Data storage device 430 is configured to store data received or generated by computing device 402, such as any of the data stored in database 410 or any outputs of processes implemented by any component of computing device 402. The gRNA generation component 440 is configured to select a plurality of candidate gRNA target sites and non-target gRNA sites within the strain sequence data as described herein. The gRNA selection component 450 is configured to analyze the plurality of candidate gRNA target sites and identify the strain-specific gRNA sequences by eliminating a portion of the plurality of candidate gRNA targets as described herein.

Communication component 460 is configured to enable communications between computing device 402 and other devices (e.g. user computing device 330 and sequencing system 310, shown in FIG. 1) over a network, such as network 350 (shown in FIG. 1), or a plurality of network connections using predefined network protocols such as TCP/IP (Transmission Control Protocol/Internet Protocol).

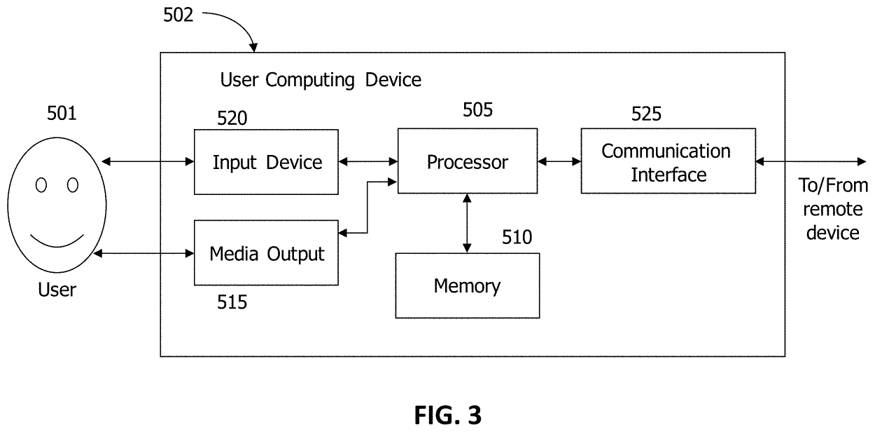

FIG. 3 depicts a configuration of a remote or user-computing device 502, such as user computing device 330 (shown in FIG. 1). Computing device 502 may include a processor 505 for executing instructions. In some aspects, executable instructions may be stored in a memory area 510. Processor 505 may include one or more processing units (e.g., in a multi-core configuration). Memory area 510 may be any device allowing information such as executable instructions and/or other data to be stored and retrieved. Memory area 510 may include one or more computer-readable media.

Computing device 502 may also include at least one media output component 515 for presenting information to a user 501. Media output component 515 may be any component capable of conveying information to user 501. In some aspects, media output component 515 may include an output adapter, such as a video adapter and/or an audio adapter. An output adapter may be operatively coupled to processor 505 and operatively coupleable to an output device such as a display device (e.g., a liquid crystal display (LCD), organic light emitting diode (OLED) display, cathode ray tube (CRT), or “electronic ink” display) or an audio output device (e.g., a speaker or headphones). In some aspects, media output component 515 may be configured to present an interactive user interface (e.g., a web browser or client application) to user 501.

In some aspects, computing device 502 may include an input device 520 for receiving input from user 501. Input device 520 may include, for example, a keyboard, a pointing device, a mouse, a stylus, a touch-sensitive panel (e.g., a touchpad or a touch screen), a camera, a gyroscope, an accelerometer, a position detector, and/or an audio input device. A single component such as a touch screen may function as both an output device of media output component 515 and input device 520.

Computing device 502 may also include a communication interface 525, which may be communicatively coupleable to a remote device. Communication interface 525 may include, for example, a wired or wireless network adapter or a wireless data transceiver for use with a mobile phone network (e.g., Global System for Mobile communications (GSM), 3G, 4G, or Bluetooth) or other mobile data network (e.g., Worldwide Interoperability for Microwave Access (WIMAX)).

Stored in memory area 510 are, for example, computer-readable instructions for providing a user interface to user 501 via media output component 515 and, optionally, receiving and processing input from input device 520. A user interface may include, among other possibilities, a web browser and client application. Web browsers enable users 501 to display and interact with media and other information typically embedded on a web page or a website from a web server. A client application allows users 501 to interact with a server application associated with, for example, a vendor or business.

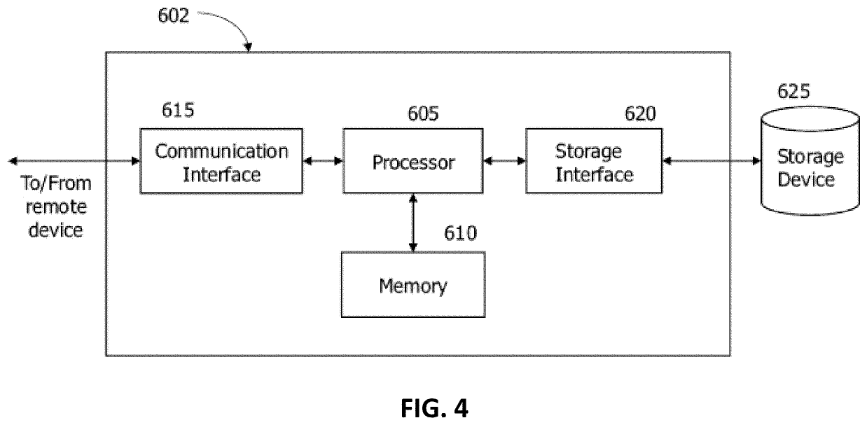

FIG. 4 illustrates an example configuration of a server system 602. Server system 602 may include, but is not limited to, database server 306 and computing device 302 (both shown in FIG. 1). In some aspects, server system 602 is similar to server system 304 (shown in FIG. 1). Server system 602 may include a processor 605 for executing instructions. Instructions may be stored in a memory area 625, for example. Processor 605 may include one or more processing units (e.g., in a multi-core configuration).

Processor 605 may be operatively coupled to a communication interface 615 such that server system 602 may be capable of communicating with a remote device such as user computing device 330 (shown in FIG. 1) or another server system 602. For example, communication interface 615 may receive requests from user computing device 330 via a network 350 (shown in FIG. 1).

Processor 605 may also be operatively coupled to a storage device 625. Storage device 625 may be any computer-operated hardware suitable for storing and/or retrieving data. In some aspects, storage device 625 may be integrated in server system 602. For example, server system 602 may include one or more hard disk drives as storage device 625. In other aspects, storage device 625 may be external to server system 602 and may be accessed by a plurality of server systems 602. For example, storage device 625 may include multiple storage units such as hard disks or solid-state disks in a redundant array of inexpensive disks (RAID) configuration. Storage device 625 may include a storage area network (SAN) and/or a network attached storage (NAS) system.

In some aspects, processor 605 may be operatively coupled to storage device 625 via a storage interface 620. Storage interface 620 may be any component capable of providing processor 605 with access to storage device 625. Storage interface 620 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 605 with access to storage device 625.

Memory areas 510 (shown in FIG. 3) and 610 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are examples only, and are thus not limiting as to the types of memory usable for storage of a computer program.

The computer systems and computer-implemented methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on vehicle or mobile devices, or associated with smart infrastructure or remote servers), and/or via computer executable instructions stored on non-transitory computer-readable media or medium.

In some aspects, a computing device is configured to implement machine learning, such that the computing device “learns” to analyze, organize, and/or process data without being explicitly programmed. Machine learning may be implemented through machine learning (ML) methods and algorithms. In one aspect, a machine learning (ML) module is configured to implement ML methods and algorithms. In some aspects, ML methods and algorithms are applied to data inputs and generate machine learning (ML) outputs. Data inputs may further include: sequencing data, sensor data, image data, video data, telematics data, authentication data, authorization data, security data, mobile device data, geolocation information, transaction data, personal identification data, financial data, usage data, weather pattern data, “big data” sets, and/or user preference data. In some aspects, data inputs may include certain ML outputs.

In some aspects, at least one of a plurality of ML methods and algorithms may be applied, which may include but are not limited to: linear or logistic regression, instance-based algorithms, regularization algorithms, decision trees, Bayesian networks, cluster analysis, association rule learning, artificial neural networks, deep learning, dimensionality reduction, and support vector machines. In various aspects, the implemented ML methods and algorithms are directed toward at least one of a plurality of categorizations of machine learning, such as supervised learning, unsupervised learning, and reinforcement learning.

In one aspect, ML methods and algorithms are directed toward supervised learning, which involves identifying patterns in existing data to make predictions about subsequently received data. Specifically, ML methods and algorithms directed toward supervised learning are “trained” through training data, which includes example inputs and associated example outputs. Based on the training data, the ML methods and algorithms may generate a predictive function that maps outputs to inputs and utilize the predictive function to generate ML outputs based on data inputs. The example inputs and example outputs of the training data may include any of the data inputs or ML outputs described above.

In another aspect, ML methods and algorithms are directed toward unsupervised learning, which involves finding meaningful relationships in unorganized data. Unlike supervised learning, unsupervised learning does not involve user-initiated training based on example inputs with associated outputs. Rather, in unsupervised learning, unlabeled data, which may be any combination of data inputs and/or ML outputs as described above, is organized according to an algorithm-determined relationship.

In yet another aspect, ML methods and algorithms are directed toward reinforcement learning, which involves optimizing outputs based on feedback from a reward signal. Specifically ML methods and algorithms directed toward reinforcement learning may receive a user-defined reward signal definition, receive a data input, utilize a decision-making model to generate an ML output based on the data input, receive a reward signal based on the reward signal definition and the ML output, and alter the decision-making model so as to receive a stronger reward signal for subsequently generated ML outputs. The reward signal definition may be based on any of the data inputs or ML outputs described above. In one aspect, an ML module implements reinforcement learning in a user recommendation application. The ML module may utilize a decision-making model to generate a ranked list of options based on user information received from the user and may further receive selection data based on a user selection of one of the ranked options. A reward signal may be generated based on comparing the selection data to the ranking of the selected option. The ML module may update the decision-making model such that subsequently generated rankings more accurately predict a user selection.

As will be appreciated based upon the foregoing specification, the above-described aspects of the disclosure may be implemented using computer programming or engineering techniques including computer software, firmware, hardware or any combination or subset thereof. Any such resulting program, having computer-readable code means, may be embodied or provided within one or more computer-readable media, thereby making a computer program product, i.e., an article of manufacture, according to the discussed aspects of the disclosure. The computer-readable media may be, for example, but is not limited to, a fixed (hard) drive, diskette, optical disk, magnetic tape, semiconductor memory such as read-only memory (ROM), and/or any transmitting/receiving medium, such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor and can be implemented in a high-level procedural and/or object-oriented programming language, and/or in assembly/machine language. As used herein, the terms “machine-readable medium” and “computer-readable medium” refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application specific integrated circuits (ASICs), logic circuits, and any other circuit or processor capable of executing the functions described herein. The above examples are examples only, and are thus not intended to limit in any way the definition and/or meaning of the term “processor.”

As used herein, the terms “software” and “firmware” are interchangeable, and include any computer program stored in memory for execution by a processor, including RAM memory, ROM memory, EPROM memory, EEPROM memory, and non-volatile RAM (NVRAM) memory. The above memory types are examples only, and are thus not limiting as to the types of memory usable for storage of a computer program.

In one aspect, a computer program is provided, and the program is embodied on a computer-readable medium. In one aspect, the system is executed on a single computer system, without requiring a connection to a server computer. In a further aspect, the system is being run in a Windows® environment (Windows is a registered trademark of Microsoft Corporation, Redmond, Washington). In yet another aspect, the system is run on a mainframe environment and a UNIX® server environment (UNIX is a registered trademark of X/Open Company Limited located in Reading, Berkshire, United Kingdom). The application is flexible and designed to run in various different environments without compromising any major functionality.

In some aspects, the system includes multiple components distributed among a plurality of computing devices. One or more components may be in the form of computer-executable instructions embodied in a computer-readable medium. The systems and processes are not limited to the specific aspects described herein. In addition, components of each system and each process can be practiced independent and separate from other components and processes described herein. Each component and process can also be used in combination with other assembly packages and processes. The present aspects may enhance the functionality and functioning of computers and/or computer systems.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Any publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches that function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Computational Design of CRISPR Guide RNAs to Enable Strain-Specific Control of Microbial Consortia

### Abstract

Microbes naturally coexist in complex, multi-strain communities. However, extracting individual microbes from and specifically manipulating the composition of these consortia remains challenging. The sequence-specific nature of CRISPR guide RNAs can be leveraged to accurately differentiate microorganisms and facilitate the creation of tools that can achieve these tasks. We developed a computational program, ssCRISPR, that designs strain-specific CRISPR guide RNA spacer sequences with user-specified target strains, protected strains, and guide RNA properties. The accuracy of the strain-specificity predictions in both Escherichia coli and Pseudomonas spp. Is verified and it is shown that up to three nucleotide mismatches are required to ensure perfect specificity. To demonstrate the functionality of ssCRISPR, computationally designed CRISPR-Cas9 guide RNAs are applied to two applications: the purification and engineering of specific microbes through one- and two-plasmid transformation workflows and the targeted removal of specific microbes using DNA-loaded liposomes. ssCRISPR will be of use in diverse microbiota engineering applications.

### Introduction

Microbes naturally co-exist in complex and dynamic communities. These microbial consortia cooperate to influence the health of the environment, domestic animals, humans, and plants. Efforts to create synthetic microbial communities have led to advances in fields including metabolic engineering and bioremediation. Numerous microbes have been extracted from natural consortia with highly specialized and essential functions. However, identifying and purifying these microbes remains challenging. Pathogens also inhabit these communities and opportunistically disrupt host health. Modern methods of removing them, including antibiotics, are highly disruptive to the survival of homeostatic, beneficial microbes and have led to the global emergence of deadly antibiotic- and bactericide-resistant pathogens. Recent advances in phage engineering and plasmid conjugation have allowed microbes to be targeted and killed in a strain-specific manner causing minimal impact on the stability of the microenvironment. Microbes have also been engineered with novel functions and introduced into natural microbiomes to improve the health of the host and engineered to selectively colonize specific microenvironments. However, exogenously provided microbes often have a difficult time penetrating consortia, finding a niche, and persisting long-term. As an alternative to supplementing microbiota with engineered microbes, microbes can instead be engineered in situ using external DNA delivery methods, increasing the endurance of the added functionality. However, methods for engineering microbes in situ often lack strain specificity, and instead introduce the exogenous DNA randomly into the microbiota.

CRISPR-Cas systems can be tuned to recognize specific genetic loci by modulating the sequence of the guide RNA (gRNA), providing opportunities for strain recognition in microbial consortia. This functionality has been harnessed for applications in strain-specific microbial engineering and elimination. Numerous programs have been developed to help design gRNAs with high cutting efficiency and low off-target cleavage rates using machine learning and deep learning models that consider the sequence and thermodynamic characteristics of the gRNA sequence. However, programs for designing gRNAs specific to individual microbial strains are lacking. One recent work achieved this goal with an effective and accessible website. However, the program lacks strain selection options, cannot be utilized for diverse CRISPR systems beyond Cas9, and defines a strain-specific gRNA as one with at least one nucleotide (nt) mismatch in the non-target strains, which has been shown to be insufficient to prevent cleavage.

In this example, a program, ssCRISPR, was created that computationally designs strain-specific CRISPR gRNAs from user-defined target and non-target strains without the common deficiencies in current programs. Genome sequences for strain options were extracted from the expansive National Center for Biotechnology Information (NCBI) genome repository, giving users over 27,000 strain selection options, or can be provided by the user. Users of ssCRISPR can also input their desired protospacer adjacent motif (PAM) sequence, target sequence length, and PAM-target orientation, giving the program the customizability required for use with any CRISPR-Cas system. Furthermore, users can select their desired criteria for specificity, from 1-4 nt, as the application will dictate the required stringency. However, it is shown that to ensure complete strain-specificity, at least 3 nt mismatches in the target sequence relative to the genomes of all non-target strains may be required. To this end, two potential applications of ssCRISPR-designed gRNAs were demonstrated: first, the purification of a single strain from a microbial consortium using a single plasmid transformation, and second, the in situ depletion of a single strain from a microbial consortium using liposomal delivery of strain-specific CRISPR-Cas9 cassettes. ssCRISPR can be downloaded and run locally either as a Python script or as an all-encompassing executable application. In either case, users can take advantage of the user-friendly graphical interface to operate the program without programming expertise.

### Results

### ssCRISPR Identifies Efficient gRNAs for Target Strains

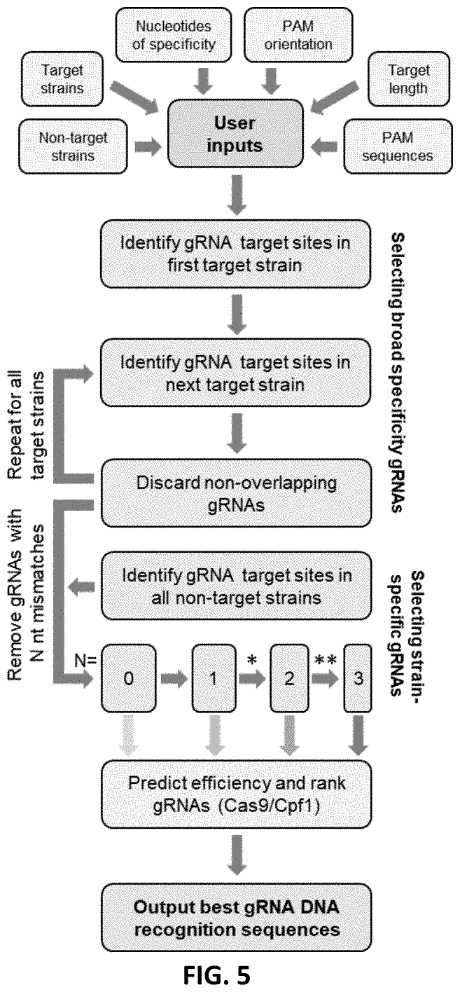

To develop ssCRISPR, a program that computationally predicts strain-specific gRNAs, a reference database of genome sequences was first needed. The NCBI genome repository was selected, which at the time of the last download included 27,569 complete bacterial genome sequences. The database is rapidly expanding to include newly sequenced genomes. The sequences can be quickly extracted from NCBI using the sequence reference number which eliminates the burdensome need to maintain the full sequences locally and allows for easy future updates. To use the repository, the table of strain names and corresponding sequence reference numbers were downloaded and the table file was packaged with the developed gRNA design program. The user then has the option to select target strains and protected, non-target strains for gRNA identification (FIG. 5). However, if a desired strain is not provided as an option, users can also provide their own sequences.

Having obtained an expansive database of strain selections, ssCRISPR was attempted to be made generalizable across any CRISPR-Cas system. To achieve this goal, user inputs for the following characteristics were created: target sequence length, PAM sequence, and PAM orientation relative to the target sequence. These inputs allow the user to apply the program to CRISPR-Cas systems ranging from Streptococcus pyogenes Cas9, which has a 20 nt target sequence, an NGG PAM sequence, and a 5′-target-PAM-3′ orientation, to E. coli Cas3, which has a 32 nt target sequence, AWG/NAG/ATG PAM sequence, and 5′-PAM-target-3′ orientation. ssCRISPR applies these criteria to sequentially search the genomes of all selected target strains for the specified PAM sequences and extract the corresponding target sequences. Native plasmids are not considered viable gRNA target sites as they may be inessential for cell survival. However, if multiple unique chromosomes exist, all are considered for possible gRNA target sites. After searching each selected strain, ssCRISPR compares the lists of identified target sequences, and only gRNA sequences with exact matches between all target strains are maintained (FIG. 5).

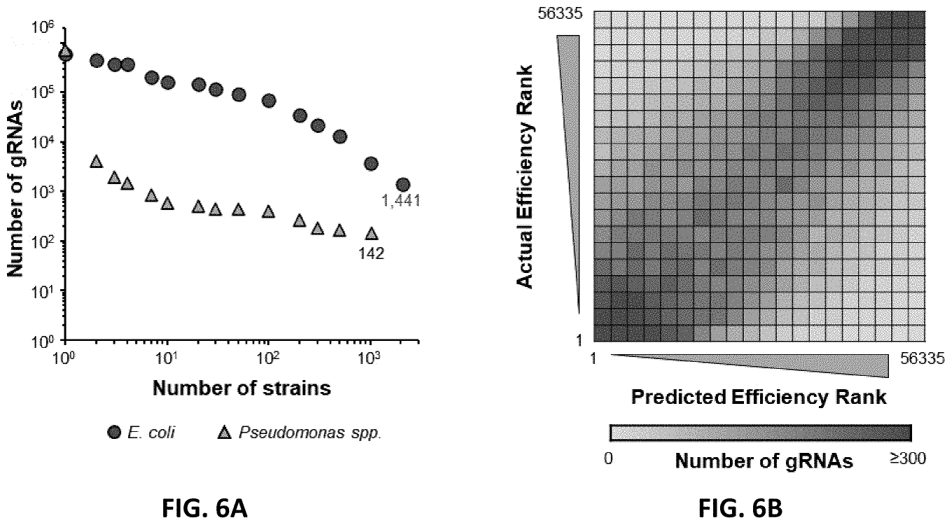

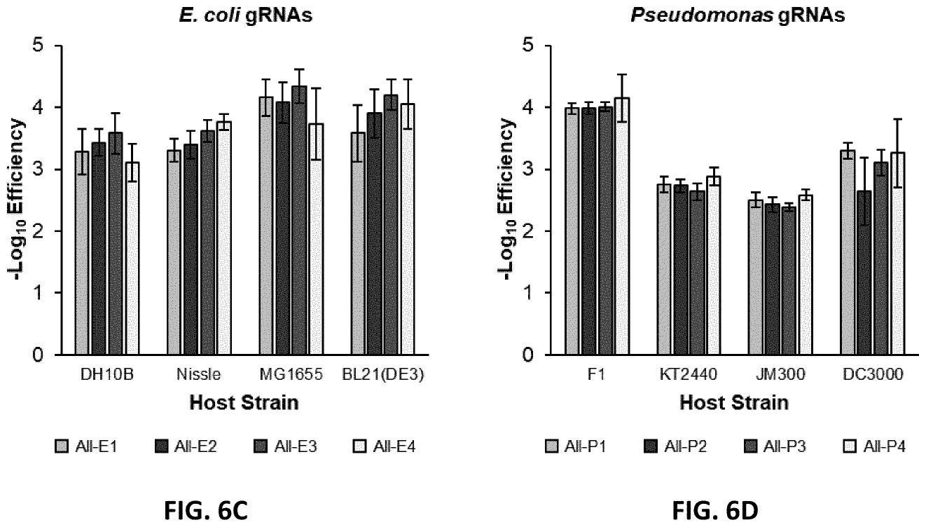

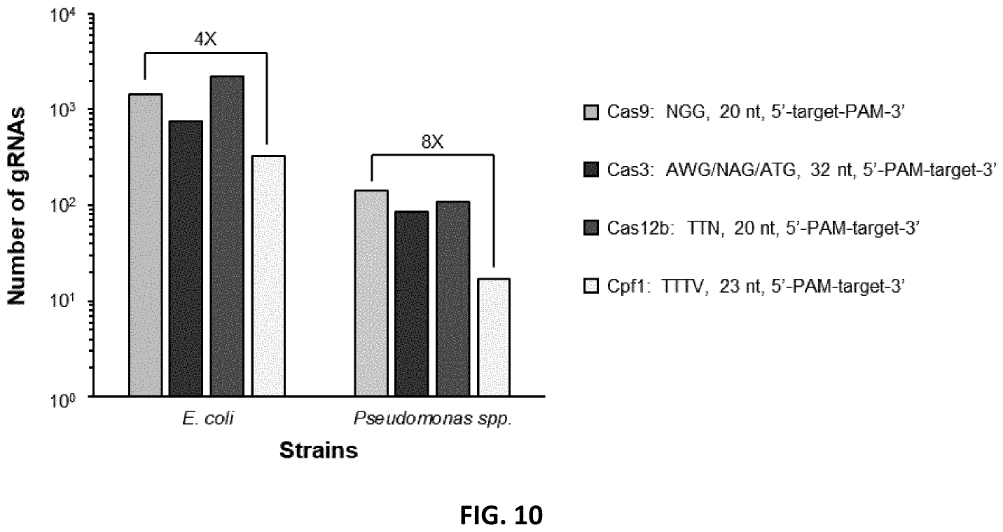

To evaluate the program, the number of CRISPR-Cas9 gRNA target sites shared between all 2,068 sequenced E. coli genomes was determined using reverse alphabetical order. ssCRISPR identified 1,441 broad-targeting E. coli gRNA sequences (FIG. 6A). The process was repeated for all 1,020 sequenced strains of Pseudomonas spp. and identified 142 total gRNA target sites. The program run for Pseudomonas spp. gRNAs eliminated viable gRNAs more rapidly than the run for E. coli, with over 99% of potential gRNAs removed after just two strains (P. zhaodongensis A252 and P. zeae OE 48.2) versus 880 E. coli strains. This observation can be explained by the larger genetic diversity between Pseudomonas species than the same-species E. coli strains. The analysis was repeated for several additional Cas proteins, including variants for Cpf1 (23 nt target length, TTTV PAM, and 5′-PAM-target-3′ orientation²⁷), Cas3 (32 nt target length, AWG, ANG, or ATG PAM, and 5′-PAM-target-3′ orientation²⁶), and Cas12b (20 nt target length, TTN PAM, and 5′-PAM-target-3′ orientation²⁸) (FIG. 10). Cas proteins with more stringent PAM sequences and longer target sequences generally had fewer potential gRNA target sites in both E. coli and Pseudomonas spp. Furthermore, Pseudomonas spp., which have a higher GC content (˜60%) than E. coli (˜50%), had a 2-fold larger reduction in the number of predicted gRNAs for Cpf1 relative to Cas9 due to the AT-rich Cpf1 PAM sequences.

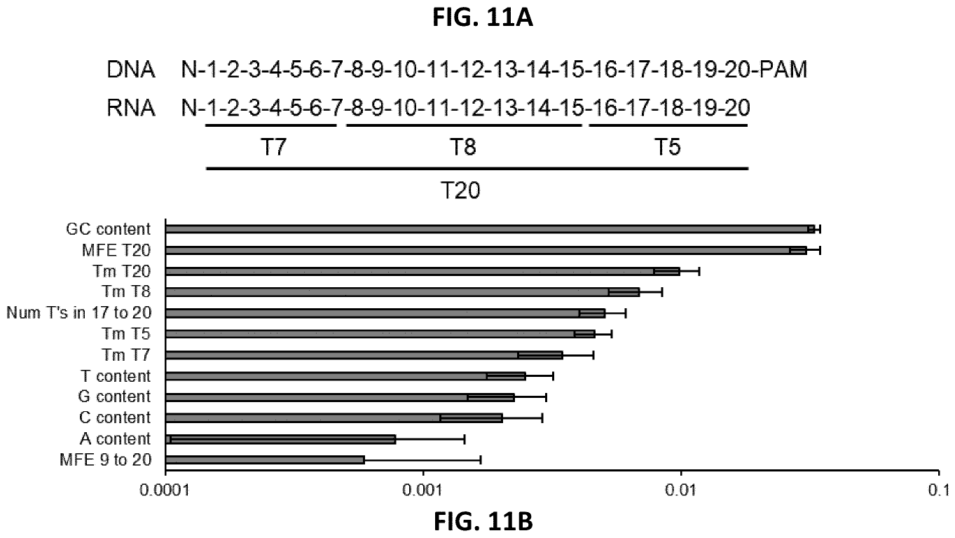

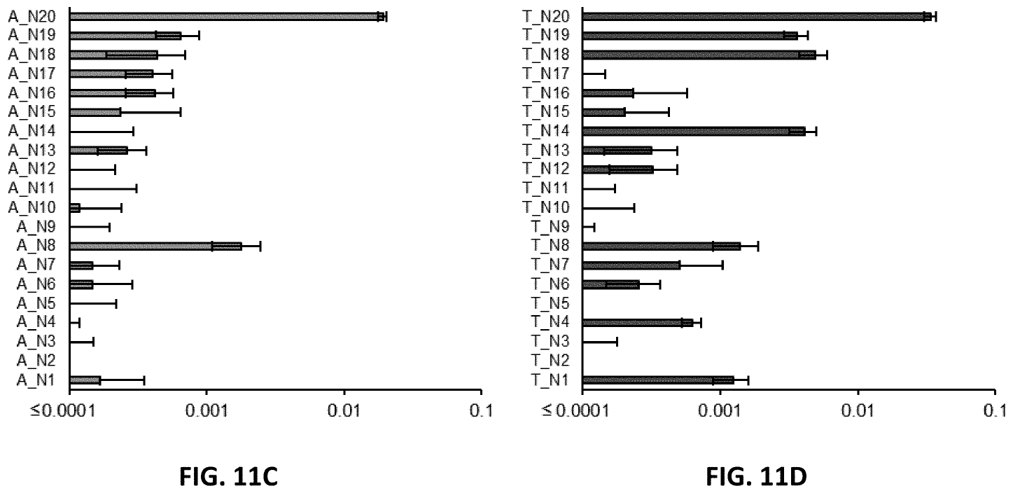

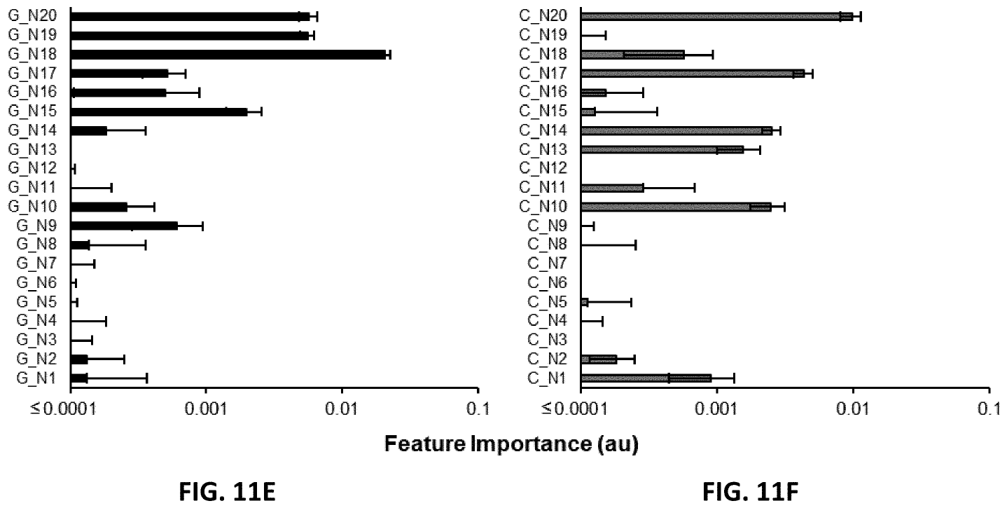

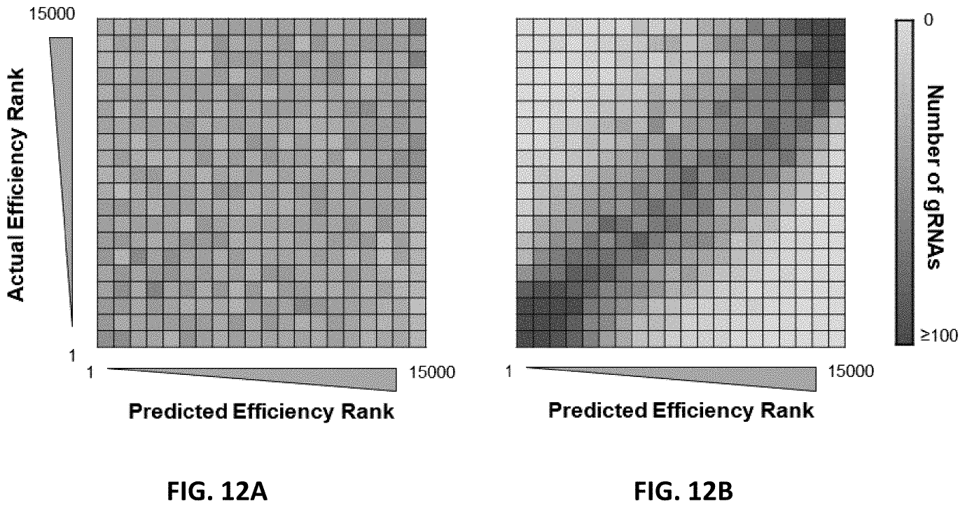

A method to select the best possible gRNAs from the list of identified sequences was searched for. To achieve this goal, a relative cleavage efficiency prediction model was adapted and incorporated. A dataset of ˜56,000 CRISPR-Cas9 gRNA sequences was used to train and optimize a gradient boosting regression machine learning model from the following 396 sequence composition and energetic properties: total A, T, C, G, and GC content, T content of the four PAM-adjacent nucleotides, presence of an A, T, C, or Gin each of the 20 PAM adjacent nucleotides (80 properties), presence of each nucleotide dimer (NN) in each of the 20 PAM adjacent nucleotides (304 properties), minimum free energy for the 12 PAM adjacent nucleotides and the full gRNA sequence, and the melting temperature for the five PAM adjacent nucleotides, next eight nucleotides, remaining nucleotides, and the full gRNA sequence. The GC content, sequence of the PAM-adjacent seed region, and thermodynamic properties of the RNA and DNA-RNA complex were found to be the most important features of the model (FIG. 11). To evaluate the accuracy of the model, we compared the predicted and actual efficiency rankings were compared (FIG. 6B). The ranking comparison displayed a strong relationship, with a Spearman's rank correlation coefficient of 0.56, which is in line with other gRNA efficiency machine learning models. To see if the model was generalizable across Cas proteins, a gRNA efficiency dataset for Cpf1 was obtained and the model was applied. The model showed no correlation between predicted and actual gRNA efficiency (FIG. 12A). As such, we used the same sequence composition and energetic properties to train a new machine-learning model for Cpf1 gRNAs. The new model showed a strong correlation, with a spearman's rank correlation coefficient of 0.57 (FIG. 12B). As such, ssCRISPR can present users with high-efficiency gRNAs for Cas9 and Cpf1. However, new models will need to be created for alternative proteins as experimental datasets become available.

To experimentally validate the program, we selected four gRNAs that target all E. coli strains and four gRNAs that target all Pseudomonas strains with the highest predicted efficiency were selected (Table 1). Plasmids for each gRNA target sequence were constructed with constitutive promoters driving gRNA expression. Next, E. coli DH10B, Nissle 1917, MG1655, and BL21(DE3) were transformed, each harboring a Cas9 expression plasmid, with a control plasmid and the gRNA plasmids. Each gRNA plasmid demonstrated a killing efficiency (see Methods) of 3- to 4-log in all four tested strains (FIG. 6C). Similar results were observed with the Pseudomonas spp. gRNAs, with killing efficiencies of 2- to 4-log achieved for each gRNA in all four strains (FIG. 2d). These results demonstrate that ssCRISPR identifies gRNAs with efficient target sites in multiple organisms.

TABLE 1
gRNA sequences

| SEQ | | |
|---|---|---|
| ID NO: | gRNA Identifier | gRNA Sequence |
| 1 | All E. coli-1 | TCGCGCGAACGCCAGACTTA |
| 2 | All E. coli-2 | TTCCTCGCCATTCTGCACGT |
| 3 | All E. coli-3 | CTTGCCGCTGATCGTTACGT |
| 4 | All E. coli-4 | GGTGTAGAACAGAATACGTT |
| 5 | All Pseudomonas-1 | CCGCCGTCGATATGAACTCT |
| 6 | All Pseudomonas-2 | CCTTAGGACCGTTATAGTTA |
| 7 | All Pseudomonas-3 | ACAAGGAATTTCGCTACCTT |
| 8 | All Pseudomonas-4 | TCGCTGACCCATTATACAAA |
| 9 | DH10B-1nt | GATACAGCACGTTTACGTGT |
| 10 | Nissle-1nt | GAGAAACAGTCGGAGCTACT |
| 11 | MG1655-1nt | ATATCGCACCCGAAGTAGTT |
| 12 | BL21(DE3)-1nt | GTTAATAGAGATCGGGCACT |
| 13 | DH10B-2nt | GTTCGTGCTTGTCTGAGTTA |
| 14 | Nissle-2nt | TATCATCCAGGAGGTGCACT |
| 15 | MG1655-2nt | ATCTTCCAGCGTAATACCTA |
| 16 | BL21(DE3)-2nt | TGGGGACAACCGAACTAACT |
| 17 | DH10B-3nt | TCGTCGTGAGCGTGAGTCAT |
| 18 | Nissle-3nt | TCGGTGAAACTCGTCTGATT |
| 19 | MG1655-3nt | TACCAACTCCCCAACTAACT |
| 20 | BL21(DE3)-3nt | CGTACTGCCTTGTAAGACGT |
| 21 | DH10B-3nt-1 | ACGCGCGATCATGAATGCGA |
| 22 | DH10B-3nt-2 | GATACAGCACGTTTACGTGT |
| 23 | DH10B-3nt-3 | GAGTTGCCACCCTGAACAGT |
| 24 | DH10B-3nt-4 | TCTCACCGGCACTGACGTTA |
| 25 | Nissle-3nt-1 | GCGGATTAATCCCAGTATTT |
| 26 | Nissle-3nt-2 | GGTCAGAGCCAGCGTGCAGT |
| 27 | Nissle-3nt-3 | CGGCCATCAAACGTGTGTTT |
| 28 | Nissle-3nt-4 | GATTTCTGCGTTCAGCGTTT |
| 29 | MG1655-3nt-1 | ATATCGCACCCGAAGTAGTT |
| 30 | MG1655-3nt-2 | ATCGGATGGCACTGTGCAAA |
| 31 | MG1655-3nt-3 | GCTCAACACCTGGCTACTTA |
| 32 | MG1655-3nt-4 | GGTTTAAGCCCGCGCACTGA |
| 33 | BL21(DE3)-3nt-1 | TCTCGGCGATGCTCTGCTTT |
| 34 | BL21(DE3)-3nt-2 | ATTAGTAGTGGCTGAACCGT |
| 35 | BL21(DE3)-3nt-3 | TCTCCGCATGACTGTGCAAA |
| 36 | BL21(DE3)-3nt-4 | CGGATCAATAACGTTACCCT |
| 37 | F1-3nt-1 | GCTGAAGCCAAGGTCACACT |
| 38 | F1-3nt-2 | GACGAAGCAATGGAGTCACT |
| 39 | F1-3nt-3 | GATGTCGATGCGGTTGTACA |
| 40 | F1-3nt-4 | GATCATCGAAGCGGTACAGT |
| 41 | KT2440-3nt-1 | GGTTCCCAAGGGATCGCACA |
| 42 | KT2440-3nt-2 | GATGCGAACCGGATTACCTT |
| 43 | KT2440-3nt-3 | GTGCGAGGAACACTGACAGT |
| 44 | KT2440-3nt-4 | TTCTCGAACCAGCGCTACTA |
| 45 | JM300-3nt-1 | GCGACACATGATCGAACACT |
| 46 | JM300-3nt-2 | CGCGTTGCACGAGTTGCTCT |
| 47 | JM300-3nt-3 | CTGGGTGACGTCGTGCATGA |
| 48 | JM300-3nt-4 | GAGTCGCGTGAAGTGAGTTT |
| 49 | DC3000-3nt-1 | CACAAATGTCGGTTTGCACT |
| 50 | DC3000-3nt-2 | GAGAAGCACGGCAAGCAAGA |
| 51 | DC3000-3nt-3 | ACTCATCAGCACGTCAGTCT |
| 52 | DC3000-3nt-4 | CAGGCGTATTTCATCACATT |
| 53 | Not Nissle | GCGTACACTGAAATCACACT |
| 54 | Enterococcus array-A1 | AATTCTGATGCCGACACTTC |
| 55 | Enterococcus array-A2 | GTATTGATGAAATTCTTGAA |
| 56 | Enterococcus array-B1 | GTTCCTCGGCACCGCGCATC |
| 57 | Enterococcus array-B2 | TTTTCATAGCTGGTCCAGTT |
| 58 | Enterococcus array-C1 | GGTAAGAGTGAAGTTCGCAG |
| 59 | Enterococcus array-C2 | TCTCGTTGAAAGCCGCCGGA |
| 60 | Enterococcus array-D1 | GGTTGGCGTCATCGTGTTCA |
| 61 | Enterococcus array-D2 | GCGTCATCGTGTTCAAGGAA |
| 62 | Enterococcus array-E1 | GAATCGTACTAAACTGGTAC |
| 63 | Enterococcus array-E2 | AATAATGAATCGTACTAAAC |
| 64 | Enterococcus array-F1 | AGCTCGTGTCGTGAGATGTT |
| 65 | Enterococcus array-F2 | CAGCTCGTGTCGTGAGATGT |
| 66 | All Pseudomonas | CACCCAGGCCAGCATGCTGC |
| | except F1 | |

### Three Nucleotide Mismatches are Required for Optimal Strain Specificity

Strain protection was then incorporated into the program by allowing the user to select non-target strains that lack the gRNA target site. However, criteria for what makes a gRNA sequence strain specific were required (FIG. 5). It has been previously demonstrated that nucleotide mismatches in the PAM and 10-12 nt PAM-adjacent seed region cause the largest reduction in cleavage efficiency. As such, a strain-specific gRNA was first defined to possess at least one nucleotide mismatch in the PAM site or the 10 nt seed region compared to all specified non-target strains. The program applied the same method described above to identify all gRNA target sequences in the specified non-target strains. Any sequence in the identified list of broad-targeting gRNAs that contained a seed region perfectly matching a gRNA from the non-target strains was removed.

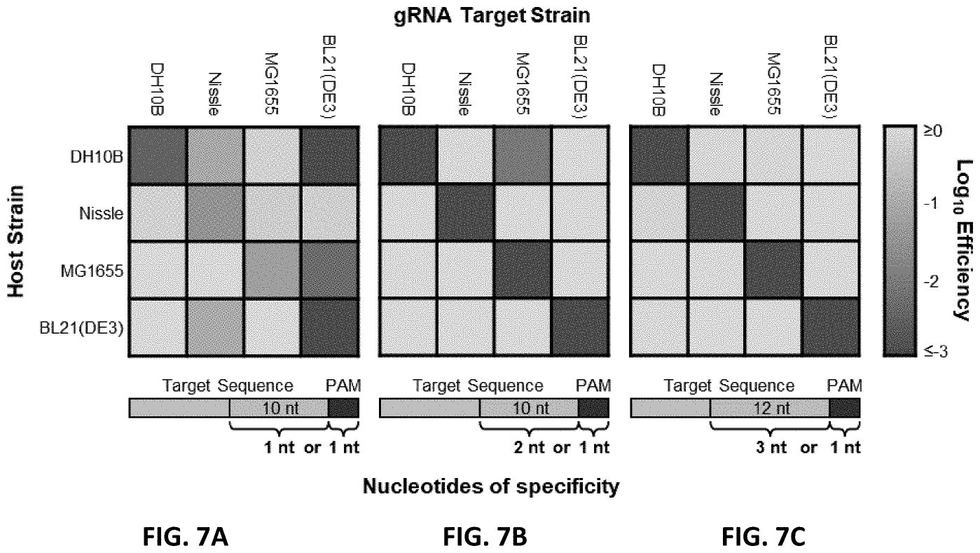

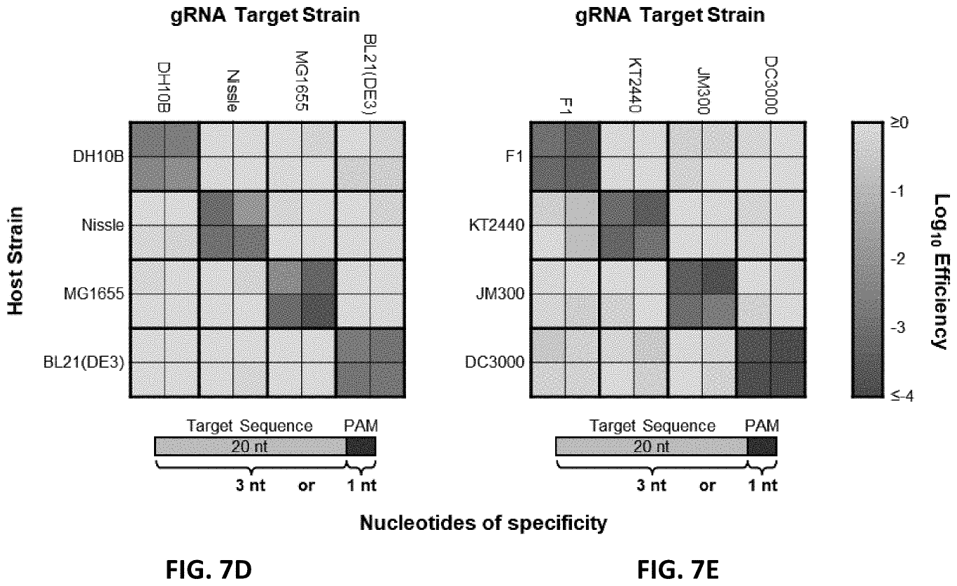

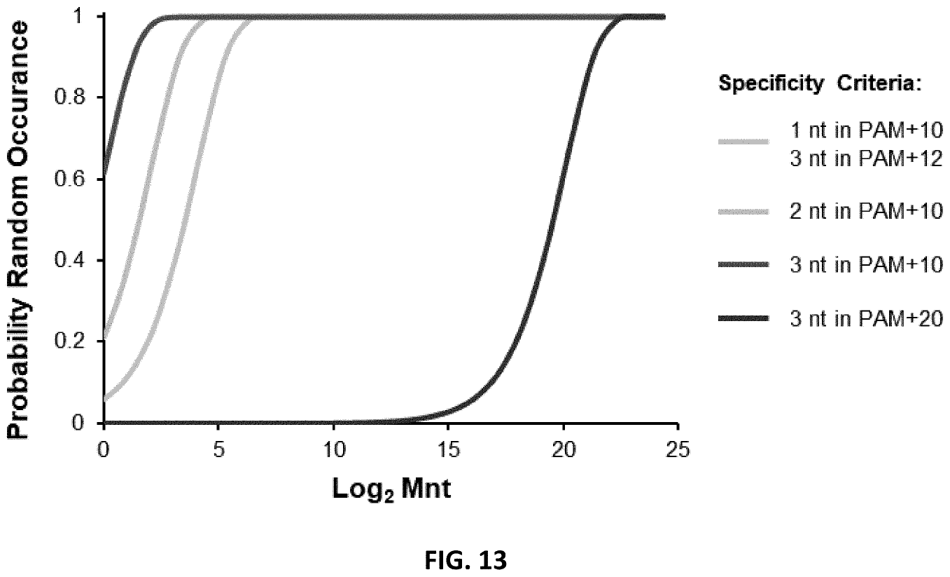

To assess this function, the efficiencies of four gRNAs were tested, one specific to each of E. coli DH10B, Nissle 1917, MG1655, and BL21(DE3), in each of the four E. coli strains. Each gRNA efficiently killed its cognate strain (FIG. 7A). However, a gRNA efficiency of greater than 1-log was also observed in 4/12 non-cognate combinations. To improve specificity and reduce the likelihood of off-target cleavage, the stringency of the program was increased to require two mismatches in the same region. Specificity was improved but remained imperfect, with all four gRNAs demonstrating efficient activity in their cognate strain and significant activity observed in 1/12 non-cognate combinations (FIG. 7B). The stringency was further increased to 3 nt but found that gRNA options were rapidly eliminated after considering each non-target strain. It was determined that requiring three mismatches in the 10 nt seed region, but ignoring the rest of the gRNA sequence, led to a high probability of each gRNA sequence occurring in any given random nucleotide sequence (FIG. 13). To alleviate this issue, the considered region was expanded to be the full 12 nt seed region. This criterion successfully identified strain-specific gRNAs, with all four tested gRNAs demonstrating efficient activity in their cognate strain and no activity in their non-cognate strains (FIG. 7C).

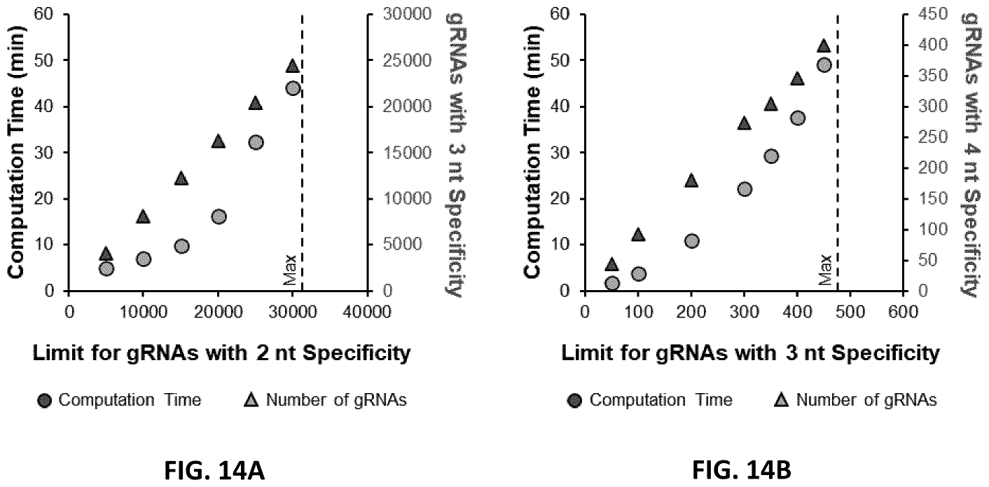

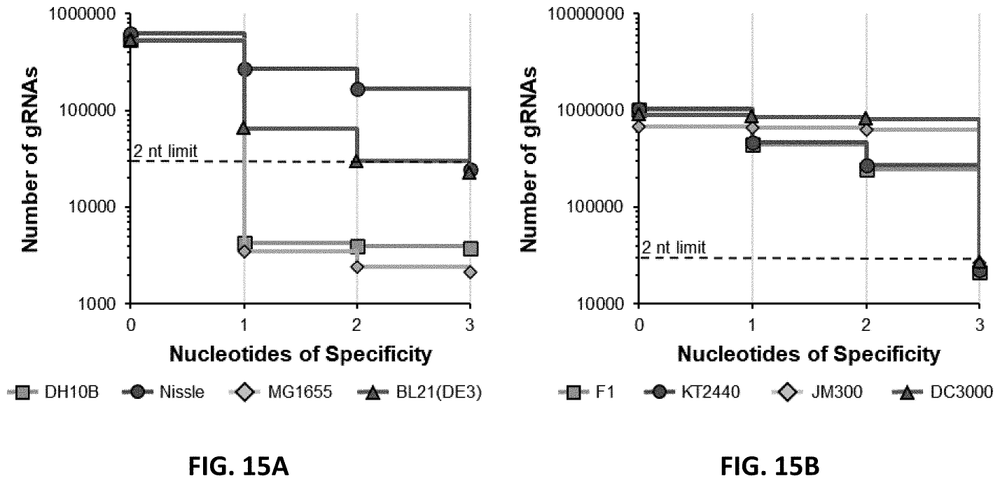

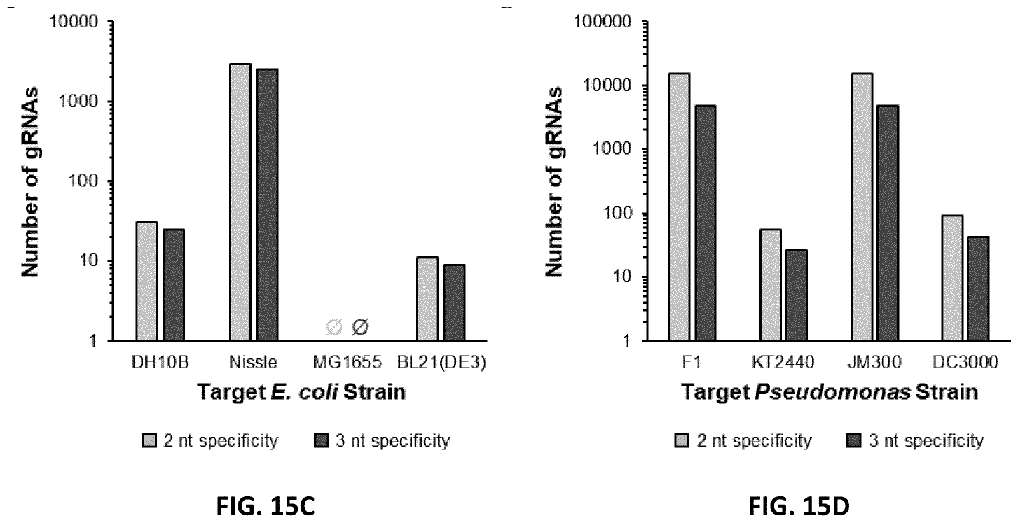

Upon further analysis, it was determined that the probability of a 12 nt gRNA seed sequence randomly occurring in any given sequence remained too high for considering many non-target strains. Specifically, 99% of gRNA sequences are eliminated by a random 80,000,000 nt sequence, corresponding to approximately 16 average-sized microbial genomes. As such, the considered region was expanded to a 20 nt target sequence. Using this criterion, over 1,000,000 strains worth of random DNA are required to eliminate 99% of gRNAs, with less than 1% of gRNAs eliminated after over 1,000 strains worth of random DNA. However, it was found that screening tens of thousands of gRNAs for 3 nt of specificity was very computationally intensive. As such, if more than 5,000 gRNAs are identified with 2 nt of specificity, 5,000 are randomly selected for further analysis (FIG. 14A). However, this number was found to be more than sufficient. ssCRISPR identified thousands of gRNAs with specificity to each of the four considered E. coli and Pseudomonas strains when the set of four was considered exclusively (FIGS. 15A and B). The number of viable gRNA sequences was reduced when all other E. coli or all other Pseudomonas strains were specified as non-target strains (FIGS. 15C and 15D, and Table 2). However, at least one gRNA was identified with 3 nt of specificity for all strains except E. coli MG1655. This result may be caused by its frequent use and analysis, as many of the sequenced strains in the reference genome database may be derived from E. coli MG1655.

TABLE 2
Target and non-target strains for determining strain-specific
gRNAs for different E. coli and Pseudomonas strains

| | Strains not | |
|---|---|---|
| Target Strains | Considered | Non-target Strains |
| E. coli DH10B | E. coli NEB10B | All other E. coli |
| E. coli Nissle 1917 | E. coli Nissle | All other E. coli |
| pZE21 | 1917 | |
| E. coli MG1655 | E. coli MG1655s, | All other E. coli |
| | tolC-, MDS | |
| E. coli BL21(DE3) | E. coli BL, Nicro, | All other E. coli |
| | C41/41(DE3), | |
| | T7/NEBexpress | |
| P. putida F1 | None | All other Pseudomonas |
| P. putida KT2440 | None | All other Pseudomonas |
| P. stutzeri JM300 | None | All other Pseudomonas |
| P. syringae pv. tomato str. | None | All other Pseudomonas |
| DC3000 | | |

The four best predicted gRNAs with specificity to each of the four E. coli strains (16 total gRNAs) were selected and tested. All 16 gRNAs maintained perfect specificity, with no significant activity observed in any non-cognate combination (FIG. 7D). To further validate the program, an additional predicted 16 strain-specific gRNAs were tested in the four Pseudomonas strains. Again, all 16 gRNAs demonstrated perfect strain-specific activity (FIG. 7E). While it was shown that 3 nt mismatches in a 20 nt gRNA target sequence allows for perfect strain specificity, ssCRISPR allows the user to specify the desired number of nucleotide mismatches (from 1-4), as fewer may be sufficient for some applications. Notably, when 4 nt of specificity are desired, the number of gRNAs with 3 nt tested is limited to 100 (FIG. 14B).

### Purifying Single Strains from Microbial Consortia Using ssCRISPR gRNAs

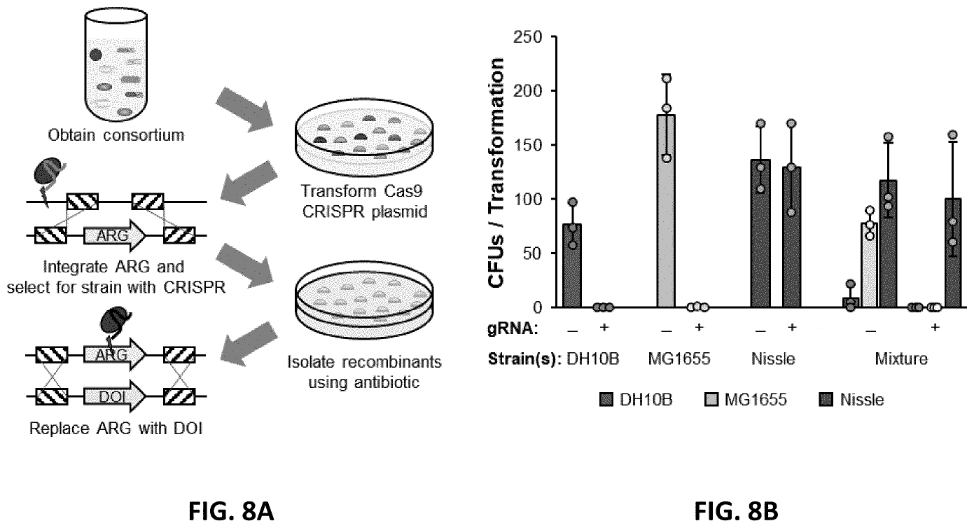

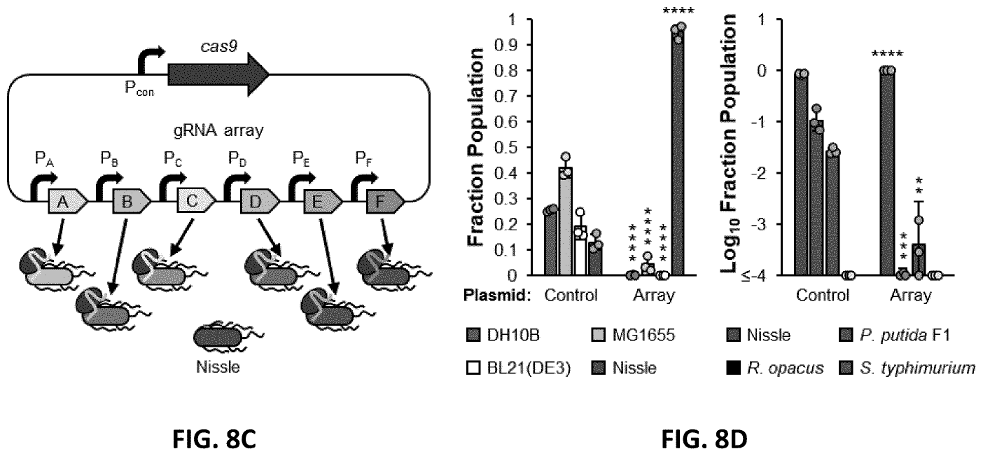

ssCRISPR was next applied to isolate and engineer a single strain from a microbial consortium. Modern methods of microbial engineering employ lambda Red-mediated recombination to engineer a strain of interest and CRISPR-Cas gRNAs that target the unmodified recombination site to select for successfully modified strains. To utilize this system to isolate and engineer microbes, a workflow was created where strain-specific gRNAs, designed using ssCRISPR, target the genomes of non-desired strains, rather than the site of recombination in the desired strain. A consortium containing the desired strain can be transformed with the Cas9/lambda Red plasmid, cultured, and transformed again with the integration cassette and strain-specific gRNA plasmid (FIG. 8A). To negate the need for a gRNA that targets the integration site, an antibiotic resistance gene can be included in the integration cassette for selection during this initial round of engineering. The antibiotic resistance gene can be later replaced with any DNA of interest using a gRNA that targets the antibiotic resistance gene. Alternatively, a two-gRNA system can be employed, where one gRNA targets the genome of non-desired strains and the second gRNA targets the engineered site. If the user wants the integration to occur in multiple strains, they can also design the second gRNA with ssCRISPR by providing a sequence file for the desired integration region in one or more of the target strains.

To validate the one-gRNA system, ssCRISPR was used to design a gRNA that protects E. coli Nissle 1917 while targeting E. coli DH10B, MG1655, and BL21(DE3). An integration cassette harboring a kanamycin resistance gene that targets the lacZ locus in E. coli Nissle 1917 was next created. The E. coli Nissle 1917 lacZ sequence is 99% homologous with the other E. coli strains, suggesting that any strain-specificity by the system would be a result of the strain-specific gRNA. The system was tested using cultures of each strain individually and in an equal-part consortium. E. coli BL21(DE3) yielded no colonies when transformed with the Cas9/lambda Red plasmid and was therefore excluded from this experiment. When the integration cassette was transformed with a control plasmid, colonies of all three strains were observed (FIG. 8B). However, in the microbial mixture, E. coli MG1655 and Nissle 1917 outcompeted E. coli DH10B due to their higher growth rates. When the strains were transformed with the strain-specific gRNA plasmid, only engineered colonies of E. coli Nissle 1917 were observed. This demonstrates that ssCRISPR can facilitate the isolation and engineering of specific microbes from a consortium. Next, the system was used to isolate and engineer E. coli Nissle 1917 from murine fecal samples. Murine fecal samples were previously obtained from mice gavaged with 10⁸ CFUs of E. coli Nissle 1917. When we transformed the Cas9/lambda Red plasmid into the fecal consortium, 100% of the resulting colonies were from E. coli Nissle 1917. This result suggests that the plasmid is not compatible with other microbial genera and can therefore be leveraged alone to purify E. coli from complex consortia.

When only strain isolation is desired, Cas9 and strain-specific gRNAs can be paired on a single plasmid, and a single transformation used to isolate the strain (FIG. 8C). Furthermore, multiple gRNAs can be expressed in an array from a single promoter and post-transcriptionally processed using intergenic RNA cleavage sites or in multiple independent and non-repetitive cassettes. To demonstrate this idea, a p15A origin plasmid was used, which only replicates in Enterococcus spp., to constitutively express Cas9 and a gRNA ELSA array. The gRNA array consisted of six non-repetitive gRNA cassettes that target different subsets of Enterococci but protect E. coli Nissle 1917 with at least 1 nt of specificity (Table 3).

TABLE 3
Strain selections for designing gRNAs in
the Enterobacteriaceae-targeting array

| gRNA Identifier | Target Strains | Non-target Strains |
|---|---|---|
| Enterococcus array-A1 | All other E. coli 0 - B | E. coli Nissle 1917 |
| and A2 | | pZE21 |
| Enterococcus array-B1 | All other E. coli C - Nico21 | E. coli Nissle 1917 |
| and B2 | | pZE21 |
| Enterococcus array-C1 | All other E. coli NMBU - Z | E. coli Nissle 1917 |
| and C2 | | pZE21 |
| Enterococcus array-D1 | All other Escherichia | E. coli Nissle 1917 |
| and D2 | All Proteus | pZE21 |
| Enterococcus array-E1 | All Enterobacter | E. coli Nissle 1917 |
| and E2 | All Klebsiella | pZE21 |
| | All Salmonella enterica | |
| Enterococcus array-F1 | All Enterococcus | E. coli Nissle 1917 |
| and F2 | All Pseudomonas | pZE21 |

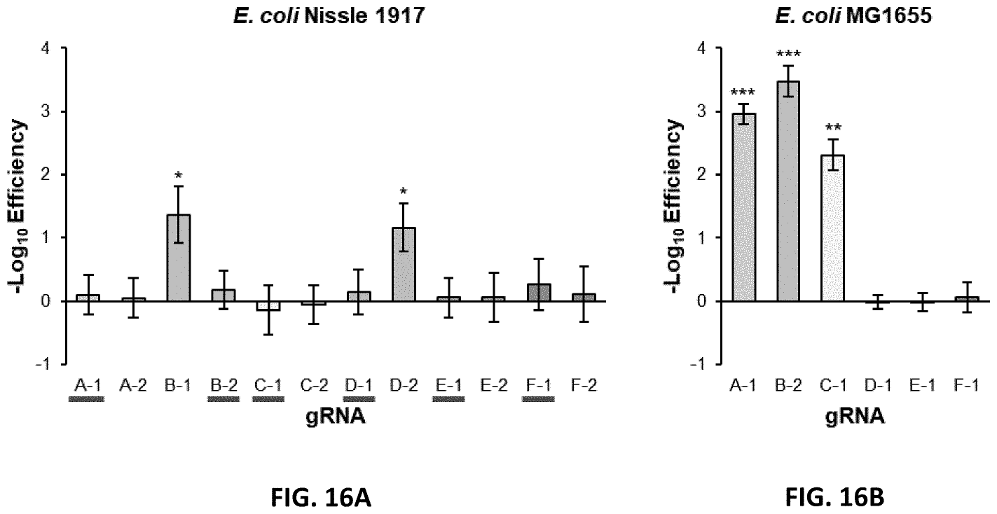

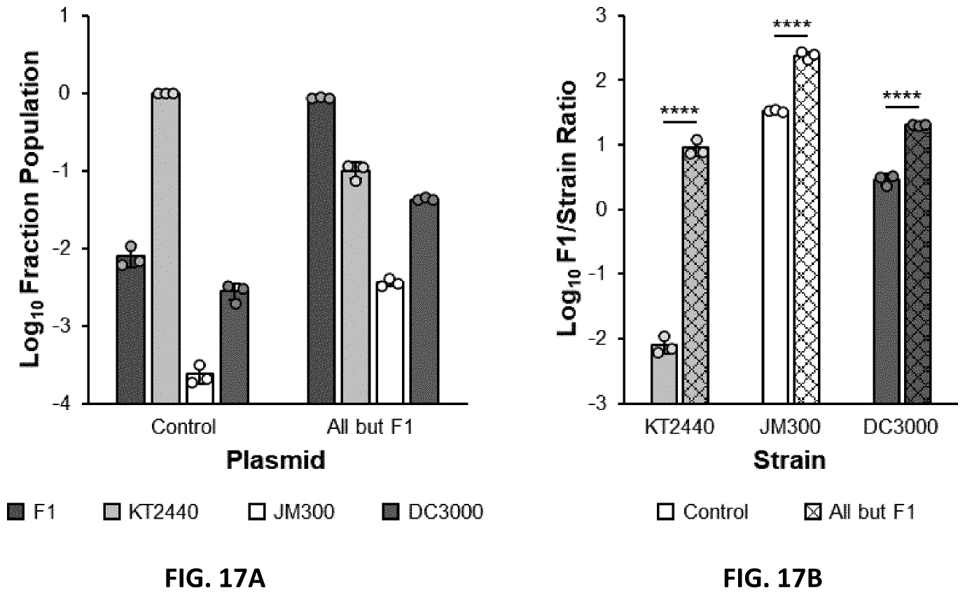

Two gRNAs from each strain group were individually tested to identify ones with the desired specificity (FIG. 16). A mixture of E. coli strains was transformed with a control plasmid and the test plasmid, a substantially higher (p<0.0001) relative abundance of E. coli Nissle 1917 was observed in the population that received the test plasmid (95%) compared to the population that received the control plasmid (13%; FIG. 8D). The same plasmid was then used to purify E. coli Nissle 1917 from a more complex strain mixture composed of P. putida F1, Salmonella typhimurium, and Rhodococcus opacus PD630. Transforming the strain mixture with the test plasmid significantly depleted P. putida F1 (p=0.0006) and S. typhimurium (p=0.0062), while increasing the abundance of E. coli Nissle 1917 (p<0.0001). R. opacus PD630, which is an incompatible host for p15A origin plasmids, was not detected after either transformation. A similar construct was created for the purification of P. putida F1 from a consortium of Pseudomonas strains, which demonstrated a strong increase (p<0.0001) in the abundance of P. putida F1 in the population that received the test plasmid (85%) compared to the population that received the control plasmid (<1%; FIG. 17). Collectively these data show that gRNAs designed using ssCRISPR can be utilized to isolate microbes from consortia in a single transformation.

### Liposome Delivery of Strain-Specific CRISPR-Cas9 Antimicrobials

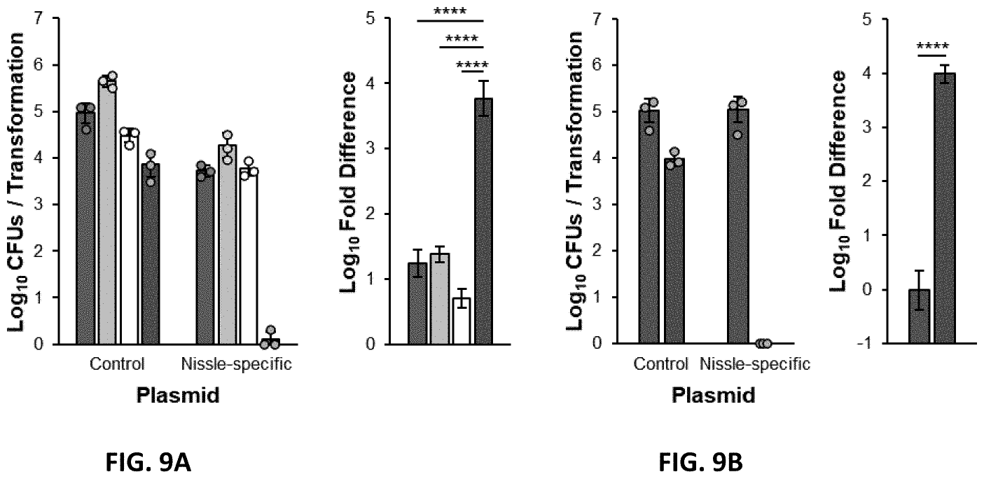

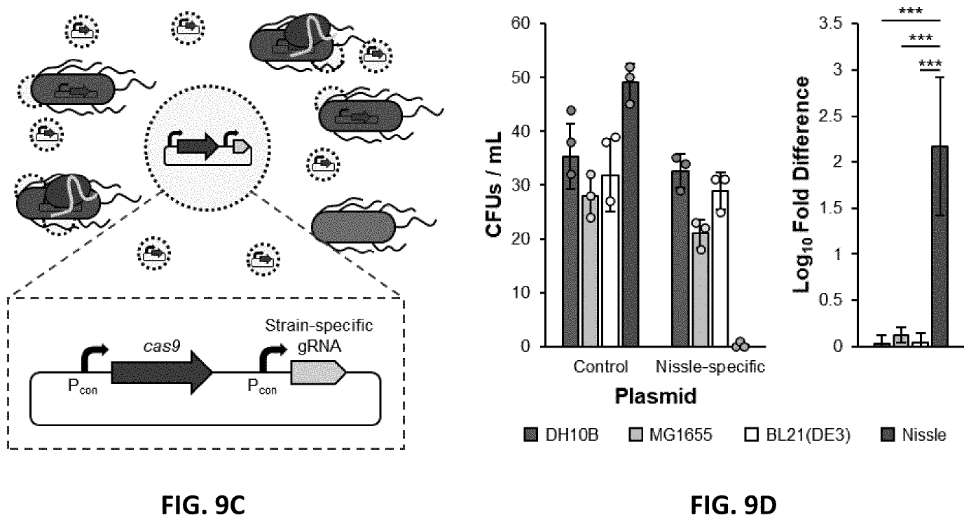

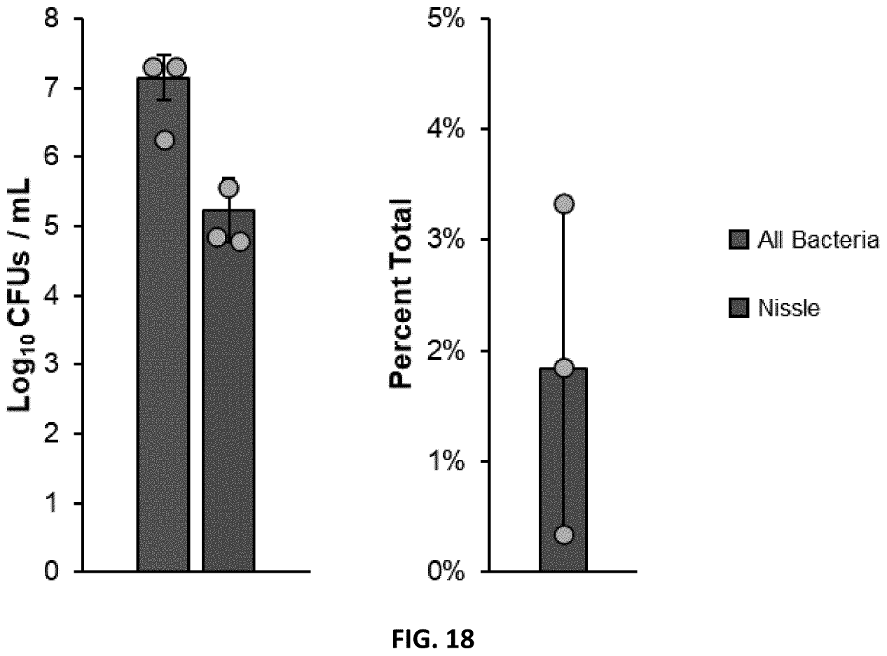

ssCRISPR also has the potential to be used to selectively remove microbes from a consortium in situ. To accomplish this goal, a gRNA that specifically targets E. coli Nissle 1917 was selected and inserted on the p15A plasmid with the constitutive Cas9 cassette. When an equal-part, multi-strain E. coli consortium was transformed with the control plasmid and test plasmid, a 3.8-log reduction was observed in E. coli Nissle 1917 CFUs for the test plasmid compared to the control plasmid treated populations (FIG. 9A). E. coli DH10B, MG1655, and BL21(DE3) also showed lower CFUs in response to transformation with the test plasmid compared to those transformed with the control plasmid, but to a significantly smaller degree than E. coli Nissle 1917 (p<0.0001). These changes may have been a result of differences in the transformation efficiency of the competent cells or plasmids. The same protocol was applied to remove E. coli Nissle 1917 from murine fecal samples. Prior to transformation, the amount of E. coli Nissle 1917 in the samples was quantified and it was determined that the strain made up approximately 2% of the aerobically-culturable microbes (FIG. 18). Transformation of the fecal consortia with the control plasmid increased the relative CFUs of E. coli Nissle to approximately 10% of the total aerobically-culturable microbes (FIG. 9B). However, the transformation of the fecal consortia with the test plasmid eliminated E. coli Nissle 1917. These results show that ssCRISPR gRNAs can be used to selectively target and eliminate microbes in consortia.

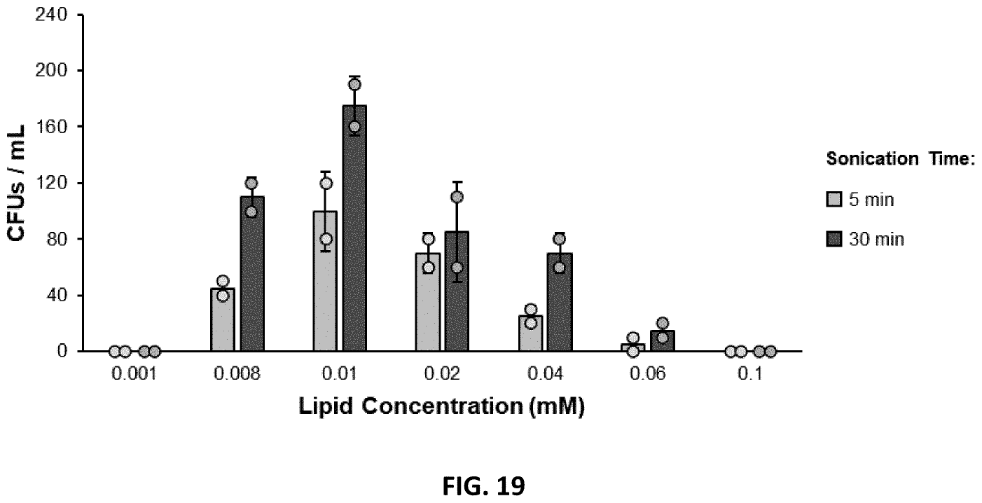

ssCRISPR gRNAs can also be used to create strain-specific CRISPR antimicrobials by pairing them with a non-specific in situ DNA delivery method. Several methods of non-specific delivery of biologics have been demonstrated in bacteria, including plasmid conjugation, bacteriophage infection, and liposome delivery. To date, bacteriophages and plasmid conjugation have been used to deliver strain-specific antimicrobials in situ. Instead, plasmid DNA carrying Cas9 and ssCRISPR gRNAs was packaged in liposomes that non-specifically fuse with microbes, and the DNA payload, which is lethal only to strains harboring the gRNA target sequence, was delivered (FIG. 9C). Liposomes were constructed and packaged post-synthesis with the control and E. coli Nissle-1917-killing test plasmid described above and the liposome synthesis and plasmid-packaging protocols were optimized (FIG. 19). An equal-part, multi-strain E. coli consortium was next incubated with the liposomes for 30 minutes and the number of cells that survived plasmid delivery was quantified (FIG. 9D). The E. coli DH10B, MG1655, and BL21(DE3) populations treated with the test and control plasmids showed similar CFUs after plasmid delivery. However, E. coli Nissle 1917 showed a 2-log reduction in viable CFUs when comparing the control and test treated populations. Together, these results show that ssCRISPR can be used to design gRNAs that target microbes in consortia with high selectivity and efficiency.

### Discussion

Manipulating microbial consortia with strain specificity can facilitate significant advances in medicine, agriculture, and climate control. However, a method for reliably distinguishing strains is essential to minimize unwanted side effects. Current programs for designing strain-specific gRNAs lack selectable strain options, cannot be customized for different CRISPR systems, and insufficiently define the characteristics that make a gRNA strain-specific. As described here, the ssCRISPR program was created to design CRISPR gRNAs with reliable strain-specific cleavage profiles. To ensure accuracy, selectivity criteria in multiple microbial strains were comprehensively tested. In addition, to allow for the wide-spread use of ssCRISPR, a wide array of user-defined parameters and more than 27,000 selectable strain options were incorporated (FIG. 5). It was shown that ssCRISPR accurately predicts gRNAs with efficient and specific activities in all selected target strains (FIG. 6) and minimal activity in selected non-target strains (FIG. 7). Furthermore, two applications of ssCRISPR were demonstrated: first, to purify specific microbes from defined consortia (FIG. 8) and second, to remove individual microbes from defined and complex consortia using broad-spectrum delivery methods such as liposomes (FIG. 9).

Purifying a specific microbe from a consortium can be a difficult task using standard modern methods such as targeted enrichment in tailored complex media and serial plating. However, this process can be simplified using strain-specific gRNAs designed with ssCRISPR. To use ssCRISPR to purify a microbe from a consortium, a degree of knowledge about the strains in the mixture is required. If the consortium is defined, designing gRNAs using ssCRISPR to target strains is a simple process. However, it is still essential that the genetic parts, such as the origin of replication and promoters, are compatible with the organisms to facilitate the purification; the origin needs to be functional in the strain of interest, and the promoters that drive expression of the Cas protein and gRNAs need to be functional in any organism with origin compatibility. Furthermore, for more complex consortia, experiments such as 16S rRNA sequencing may be required to first characterize the composition of the mixture and identify relevant strains. However, the isolation process can be improved by carefully selecting origins with narrow compatibility groups (FIG. 8D) and by selecting growth conditions favorable for the desired microbe.

Creating technologies to remove specific microbes from a consortium is essential to combat the growing issues of antibiotic- and bactericide-resistant pathogens in domesticated animals, humans, and plants. Identifying gRNAs for strain-specific removal is simpler than for purification, as microbial diversity becomes an advantage. For this application, genetic parts only need to be functional in the selected target strains. However, for the delivery of strain-specific CRISPR antimicrobials, factors including delivery efficiency and genetic remnants need to be considered. Recent advances in plasmid conjugation allow for significantly higher transfer and delivery rates of the CRISPR cassettes. However, genetic material transferred via bacteriophages, viral vectors, and plasmid conjugation is permanent once introduced into the environment, and widespread delivery of this replicating genetic material into native microbes can have adverse biological consequences. Here, as a proof of concept, plasmid-packaged liposomes were used to deliver the CRISPR payload but a low uptake efficiency was observed. However, liposomes have the potential to deliver antimicrobial CRISPR systems in non-permanent forms, including in RNA and protein forms, that are degraded intracellularly. Furthermore, RNA- and protein-based payloads may have a higher delivery efficiency than plasmids when packaged in liposomes, as both can be engineered to penetrate a cell membrane more easily than plasmids in the event that the liposome only fuses with the outer membrane.

The ssCRISPR program is not without limitations. The selectable strain options in ssCRISPR are derived from the NCBI genome repository and can be easily updated to include the rapidly accumulating new microbial genomes. However, the number of strains with sequenced genomes pales in comparison to the predicted 10¹² microbial species predicted to exist on Earth. As such, the true specificity of the gRNAs designed by the program will never be completely defined until all microbial genomes have been sequenced. In addition, although the ssCRISPR efficiency predictions for Cas9 and Cpf1 gRNAs are comparable to numerous other machine learning models, they fall behind recent deep learning models in accuracy. Fortunately, in most applications of ssCRISPR, only a highly active gRNA, rather than the best gRNA, is needed. To this end, when considering the top 5% most efficient gRNAs in a defined group, ssCRISPR predicts 96% (Cas9) or 98% (Cpf1) of the subset to be above the true median efficiency (FIG. 6B and FIG. 12B). Therefore, ssCRISPR efficiency predictions are sufficient to select for highly effective gRNAs.

In summary, ssCRISPR was developed, a user-friendly program for computationally designing strain-specific gRNAs for diverse microbes and CRISPR systems. The computational tool was validated by testing gRNAs with a wide array of target and non-target strain profiles in E. coli and Pseudomonas spp. Furthermore, two applications of the program were demonstrated, including the strain-specific isolation and removal of individual microbes from consortia. However, the program can facilitate numerous additional applications in microbiome engineering in humans and the environment. ssCRISPR is easily accessible and can be downloaded and run locally as a Python script or as a single package executable application without programming knowledge through the user interface. ssCRISPR will be a valuable tool for managing the health of livestock, plants, and humans, identifying microbes with novel characteristics, exploring the dynamics of microbial communities, and tailoring microbiota for improved functions.

### Methods

### Generating Strain Selection Options and Obtaining Genome Sequences

All programming was performed using Python 3.7, Spyder IDE, and Anaconda software packages. A list of bacterial strain names and sequence reference numbers was downloaded from NCBI (https://www.ncbi.nlm.nih.gov/genome/browse#!/prokaryotes/). Strains were filtered for complete genomes to remove partial or incomplete sequences and for bacteria to remove archaea. The list was then imported into the Python program. To create selectable strain choices, the list was sorted alphabetically, and duplicates were removed, only retaining the first sequence in the downloaded list. Genome sequences for the selected target and non-target strains are then individually extracted from the NCBI server using Entrez.efetch and the genome reference numbers. To account for short temporary lapses in the NCBI servers, genome calls are attempted 10 times before drawing an error.

### Identifying Strain-Specific Guide RNAs

To generate gRNAs with target sites in all selected target strains, genome sequences are individually extracted from the NCBI database. Locations of all PAM sites are then identified in the genome of the first selected target strain. Next, the specified number of PAM adjacent nucleotides are extracted with the specified orientation relative to the PAM site to generate a string with the gRNA sequence. All identified gRNA sequences are compiled in a list. This gRNA target site identification process is then repeated for the second selected target strain. The two lists of gRNA sequences are then compared and only sequences present in both lists are maintained. This process is repeated for all remaining target strains to generate a list of gRNA sequences, termed here as perfect gRNAs, present in all selected target strains with perfect homology.

To protect strains from gRNA cleavage, the program extracts genome sequences from the NCBI database in batches of 25 strains. Locations for the PAM sequences are then identified from the combined genomes and the respective gRNA sequences are extracted and compiled in a list of non-target strain gRNAs. To generate a list of strain-specific gRNAs, gRNA sequences shared between the perfect gRNAs list and the non-target gRNAs list are first removed from the list of perfect gRNAs, resulting in a list of gRNAs with at least 1 nt of specificity. If additional nucleotides of specificity are required, the remaining list of perfect gRNAs is sequentially input into functions that generate lists of all sequence permutations with 1, 2, and 3 nt mismatches, and the shared sequences are removed from the list of perfect gRNAs until the desired degree of specificity is reached.

### Predicting Relative Guide RNA Cleavage Efficiencies

We altered a method of gRNA efficiency predictions previously described by Guo et al was altered. The set of 56,335 Cas9 gRNA sequences assessed by Guo et al. and 15,000 Cpf1 gRNA sequences assessed by Kim et al. were independently analyzed for the following 396 sequence composition and energetic properties: total A, T, C, G, and GC content, T content of the four PAM-adjacent nucleotides, presence of an A, T, C, or G in each of the 20 PAM adjacent nucleotides (80 properties), presence of each nucleotide dimer (NN) in each of the 20 PAM adjacent nucleotides (304 properties), minimum free energy for the 12 PAM adjacent nucleotides and the full gRNA sequence, and the melting temperature for the five PAM adjacent nucleotides, next eight nucleotides, remaining nucleotides, and the full gRNA sequence. The resulting property array and the corresponding experimental gRNA cleavage rates were used to train gradient boosting regression machine learning models with a 90:10 split between the training group and test group. The models were optimized by tuning the following parameters until the minimum sum squared error was reached for the test groups: the number of boosting stages, the minimum number of samples required to split an internal node, the maximum depth of the tree, and the learning rate.

### Plasmids, Strains, and Growth Conditions

The Pseudomonas pCas9-RK2K and pSEVA-gRNAT plasmids were purchased from GenScript (catalog numbers MC_0000261 and MC_0000262). Plasmids were designed using SnapGene and assembled in E. coli DH10B using the Gibson Assembly (100 mM Tris-HCl, 10 mM MgCl₂, 0.2 mM dNTPs, 10 mM DTT, 5% PEG-8000, 1 mM NAD⁺, 4 U/μL Taq DNA ligase, 4 U/mL T5 exonuclease, 25 U/mL Phusion DNA polymerase) or Golden Gate Assembly (1×T4 ligase buffer, 1×Cutsmart buffer, 40 U/μL T4 ligase, 1 U/μL SapI, 1 U/μL DpnI) methods. Plasmids lethal to E. coli DH10B were instead assembled in E. coli Nissle 1917. Plasmids harboring both Cas9 and gRNA expression cassettes were assembled in strains expressing AttJ, a TetR-like transcription factor, to repress the P_{attKLM}-cas9 cassette and minimize toxicity. Plasmid DNA was isolated using the PureLink Quick Plasmid Miniprep Kit (K210011, Invitrogen) or PureLink HiPure Plasmid Midiprep Kit (K210005, Invitrogen), and polymerase chain reaction (PCR) products were extracted from electrophoresis gels using the Zymoclean Gel DNA Recovery Kit (D4008, ZYMO research). Chemicals were purchased from Millipore Sigma (Burlington, MA, USA). Enzymes were purchased from New England Biolabs (Ipswich, MA, USA). All Sanger and next-generation sequencing was performed by Genewiz (South Plainfield, NJ, USA). Primers were purchased from Integrated DNA Technologies (Coralville, IA, USA). All plasmids and parts constructed and used in this work are summarized in Tables 4 and 5, respectively.

TABLE 4
Plasmids used

| | | | |
|---|---|---|---|
| Plasmid | | | Antibiotic |
| Name | Genetic Parts | Origin | Resistance |

Recombineering

| | | | |
|---|---|---|---|
| pMP11 | Pcon-cas9 + pBAD-λ Red genes + | oriR101 | Ampicillin |
| | Ptet-gRNA-pBR322ori | | |
| pAGR540 | Pcon-kanR lacZ integration | p15A | Spectinomycin |
| pCAS- | Pcon-gRNA | oriV | Gentamycin |
| RKTK | | | |
| pSEVA- | Pcon-cas9 + pBAD-λ Red genes + | pBR322 | Tetracycline |
| gRNAT | PrhaB-gRNA-PBR322ori + Pcon-sacB | | |

gRNA Characterization

| | | | |
|---|---|---|---|
| pAGR287 | Pcon-cas9 | pSC101 | Kanamycin |
| pAGR516 | Ptet-gRNA_All E. coli-1 | p15A | Spectinomycin |
| PAGR517 | Ptet-gRNA_All E. coli-2 | p15A | Spectinomycin |
| pAGR518 | Ptet-gRNA_All E. coli-3 | p15A | Spectinomycin |
| pAGR519 | Ptet-gRNA_All E. coli-4 | p15A | Spectinomycin |
| pSV018 | Pcon-gRNA_All Pseudomonas-1 | pBR322 | Gentamycin |
| pSV019 | Pcon-gRNA_All Pseudomonas-2 | pBR322 | Gentamycin |
| pSV020 | Pcon-gRNA_All Pseudomonas-3 | pBR322 | Gentamycin |
| pSV021 | Pcon-gRNA_All Pseudomonas-4 | pBR322 | Gentamycin |
| pAGR480 | Ptet-gRNA_DH10B-1nt | p15A | Spectinomycin |
| pAGR482 | Ptet-gRNA_Nissle-1nt | p15A | Spectinomycin |
| pAGR483 | Ptet-gRNA_MG1655-1nt | p15A | Spectinomycin |
| pAGR484 | Ptet-gRNA_BL21(DE3)-1nt | p15A | Spectinomycin |
| pAGR500 | Ptet-gRNA_DH10B-2nt | p15A | Spectinomycin |
| pAGR502 | Ptet-gRNA_Nissle-2nt | p15A | Spectinomycin |
| pAGR503 | Ptet-gRNA_MG1655-2nt | p15A | Spectinomycin |
| pAGR504 | Ptet-gRNA_BL21(DE3)-2nt | p15A | Spectinomycin |
| pAGR505 | Ptet-gRNA_DH10B-3nt | p15A | Spectinomycin |
| pAGR507 | Ptet-gRNA_Nissle-3nt | p15A | Spectinomycin |
| pAGR508 | Ptet-gRNA_MG1655-3nt | p15A | Spectinomycin |
| pAGR509 | Ptet-gRNA_BL21(DE3)-3nt | p15A | Spectinomycin |
| pAGR520 | Ptet-gRNA_DH10B-3nt-1 | p15A | Spectinomycin |
| pAGR521 | Ptet-gRNA_DH10B-3nt-2 | p15A | Spectinomycin |
| pAGR522 | Ptet-gRNA_DH10B-3nt-3 | p15A | Spectinomycin |
| pAGR523 | Ptet-gRNA_DH10B-3nt-4 | p15A | Spectinomycin |
| pAGR524 | Ptet-gRNA_Nissle-3nt-1 | p15A | Spectinomycin |
| pAGR525 | Ptet-gRNA_Nissle-3nt-2 | p15A | Spectinomycin |
| pAGR526 | Ptet-gRNA_Nissle-3nt-3 | p15A | Spectinomycin |
| pAGR527 | Ptet-gRNA_Nissle-3nt-4 | p15A | Spectinomycin |
| pAGR528 | Ptet-gRNA_MG1655-3nt-1 | p15A | Spectinomycin |
| pAGR529 | Ptet-gRNA_MG1655-3nt-2 | p15A | Spectinomycin |
| pAGR530 | Ptet-gRNA_MG1655-3nt-3 | p15A | Spectinomycin |
| pAGR531 | Ptet-gRNA_MG1655-3nt-4 | p15A | Spectinomycin |
| pAGR532 | Ptet-gRNA_BL21(DE3)-3nt-1 | p15A | Spectinomycin |
| pAGR533 | Ptet-gRNA_BL21(DE3)-3nt-2 | p15A | Spectinomycin |
| pAGR534 | Ptet-gRNA_BL21(DE3)-3nt-3 | p15A | Spectinomycin |
| pAGR535 | Ptet-gRNA_BL21(DE3)-3nt-4 | p15A | Spectinomycin |
| pAGR548 | Pcon-gRNA_F1-3nt-1 | pBR322 | Gentamycin |
| pAGR549 | Pcon-gRNA_F1-3nt-2 | pBR322 | Gentamycin |
| pAGR550 | Pcon-gRNA_F1-3nt-3 | pBR322 | Gentamycin |
| pAGR551 | Pcon-gRNA_F1-3nt-4 | pBR322 | Gentamycin |
| pAGR552 | Pcon-gRNA_KT2440-3nt-1 | pBR322 | Gentamycin |
| pAGR553 | Pcon-gRNA_KT2440-3nt-2 | pBR322 | Gentamycin |
| pAGR554 | Pcon-gRNA_KT2440-3nt-3 | pBR322 | Gentamycin |
| pAGR555 | Pcon-gRNA_KT2440-3nt-4 | pBR322 | Gentamycin |
| pAGR556 | Pcon-gRNA_JM300-3nt-1 | pBR322 | Gentamycin |
| pAGR557 | Pcon-gRNA_JM300-3nt-2 | pBR322 | Gentamycin |
| pAGR558 | Pcon-gRNA_JM300-3nt-3 | pBR322 | Gentamycin |
| pAGR559 | Pcon-gRNA_JM300-3nt-4 | pBR322 | Gentamycin |
| pAGR560 | Pcon-gRNA_DC3000-3nt-1 | pBR322 | Gentamycin |
| pAGR561 | Pcon-gRNA_DC3000-3nt-2 | pBR322 | Gentamycin |
| pAGR562 | Pcon-gRNA_DC3000-3nt-3 | pBR322 | Gentamycin |
| pAGR563 | Pcon-gRNA_DC3000-3nt-4 | pBR322 | Gentamycin |

Strain-specific Purification

| | | | |
|---|---|---|---|
| pAGR544 | Ptet-gRNA_Not Nissle | p15A | Spectinomycin |
| pAGR564 | Ptet-gRNA_Enterococcus array-A1 | p15A | Spectinomycin |
| pAGR565 | Ptet-gRNA_Enterococcus array-A2 | p15A | Spectinomycin |
| pAGR566 | Ptet-gRNA_Enterococcus array-B1 | p15A | Spectinomycin |
| pAGR567 | Ptet-gRNA_Enterococcus array-B2 | p15A | Spectinomycin |
| pAGR568 | Ptet-gRNA_Enterococcus array-C1 | p15A | Spectinomycin |
| pAGR569 | Ptet-gRNA_Enterococcus array-C2 | p15A | Spectinomycin |
| pAGR570 | Ptet-gRNA_Enterococcus array-D1 | p15A | Spectinomycin |
| pAGR571 | Ptet-gRNA_Enterococcus array-D2 | p15A | Spectinomycin |
| pAGR572 | Ptet-gRNA_Enterococcus array-E1 | p15A | Spectinomycin |
| pAGR573 | Ptet-gRNA_Enterococcus array-E2 | p15A | Spectinomycin |
| pAGR574 | Ptet-gRNA_Enterococcus array-F1 | p15A | Spectinomycin |
| pAGR575 | Ptet-gRNA_Enterococcus array-F2 | p15A | Spectinomycin |
| pAGR578 | Ptet-gRNA_Enterococcus array full | p15A | Spectinomycin |
| pAGR620 | Ptet-gRNA_Enterococcus array full + | p15A | Spectinomycin |
| | PattKLM-cas9 | | |
| pAGR649 | pSEVA-gRNA_All Pseudomonas | pBR322 | Gentamycin |
| | except F1 + PattKLM-cas9 | | |

Strain-specific Removal

| | | | |
|---|---|---|---|
| pAGR619 | Ptet-gRNA_Nissle-1 + PattKLM-cas9 | p15A | Spectinomycin |

Controls

| | | | |
|---|---|---|---|
| pAGR129 | Empty vector - E. coli | p15A | Spectinomycin |
| pSV022 | Empty vector - Pseudomonas | pBR322 | Gentamycin |
| pAGR632 | Pcon-attJ | pSC101 | Kanamycin |

TABLE 5
Genetic parts used

| SEQ ID NO: | Part | Nucleotide Sequence |
|---|---|---|
| 67 | gRNA Cas9 | gttttagagctagaaatagcaagttaaaataaggctagtccgttatcaacttgaaaaagtggc |
| | handle and | accgagtcggtgctttttttgaagcttgggcccgaacaaaaactcatctcagaagaggatct |
| | terminator | gaatagcgccgtcgaccatcatcatcatcatcattgagtttaaacggtctccagcttggctgtt |
| | | ttggcggatgagagaagattttcagcctgatacagattaaatcagaacgcagaagcggtct |
| | | gataaaacagaatttgcctggcggcagtagcgcggtggtcccacctgaccccatgccgaa |
| | | ctcagaagtgaaacgccgtagcgccgatggtagtgtggggtctccccatgcgagagtag |
| | | ggaactgccaggcatcaaataaaacgaaaggctcagtcgaaagactgggcctttcgtttta |
| | | tctgttgtttgtcggtgaac |
| 68 | E. coli cas9 | tttacggctagctcagtcctaggtactatgctagctatcagcaggacgcactgaccaggag |
| | with | gtacaatcaATGgataagaaatactcaataggcttagatatcggcacaaatagcgtcgg |
| | promoter | atgggcggtgatcactgatgaatataaggttccgtctaaaaagttcaaggttctgggaaata |
| | and RBS | cagaccgccacagtatcaaaaaaaatcttataggggctcttttatttgacagtggagagaca |
| | | gcggaagcgactcgtctcaaacggacagctcgtagaaggtatacacgtcggaagaatcgt |
| | | atttgttatctacaggagattttttcaaatgagatggcgaaagtagatgatagtttctttcatcga |
| | | cttgaagagtcttttttggtggaagaagacaagaagcatgaacgtcatcctatttttggaaata |
| | | tagtagatgaagttgcttatcatgagaaatatccaactatctatcatctgcgaaaaaaattggt |
| | | agattctactgataaagcggatttgcgcttaatctatttggccttagcgcatatgattaagtttc |
| | | gtggtcattttttgattgagggagatttaaatcctgataatagtgatgtggacaaactatttatcc |
| | | agttggtacaaacctacaatcaattatttgaagaaaaccctattaacgcaagtggagtagatg |
| | | ctaaagcgattctttctgcacgattgagtaaatcaagacgattagaaaatctcattgctcagct |
| | | ccccggtgagaagaaaaatggcttatttgggaatctcattgctttgtcattgggtttgacccct |
| | | aattttaaatcaaattttgatttggcagaagatgctaaattacagctttcaaaagatacttacgat |
| | | gatgatttagataatttattggcgcaaattggagatcaatatgctgatttgtttttggcagctaa |
| | | gaatttatcagatgctattttactttcagatatcctaagagtaaatactgaaataactaaggctc |
| | | ccctatcagcttcaatgattaaacgctacgatgaacatcatcaagacttgactcttttaaaagc |
| | | tttagttcgacaacaacttccagaaaagtataaagaaatcttttttgatcaatcaaaaaacgga |
| | | tatgcaggttatattgatgggggagctagccaagaagaattttataaatttatcaaaccaatttt |
| | | agaaaaaatggatggtactgaggaattattggtgaaactaaatcgtgaagatttgctgcgca |
| | | agcaacggacctttgacaacggctctattccccatcaaattcacttgggtgagctgcatgct |
| | | attttgagaagacaagaagacttttatccatttttaaaagacaatcgtgagaagattgaaaaa |
| | | atcttgacttttcgaattccttattatgttggtccattggcgcgtggcaatagtcgttttgcatgg |
| | | atgactcggaagtctgaagaaacaattaccccatggaattttgaagaagttgtcgataaagg |
| | | tgcttcagctcaatcatttattgaacgcatgacaaactttgataaaaatcttccaaatgaaaaa |
| | | gtactaccaaaacatagtttgctttatgagtattttacggtttataacgaattgacaaaggtcaa |
| | | atatgttactgaaggaatgcgaaaaccagcatttctttcaggtgaacagaagaaagccattg |
| | | ttgatttactcttcaaaacaaatcgaaaagtaaccgttaagcaattaaaagaagattatttcaa |
| | | aaaaatagaatgttttgatagtgttgaaatttcaggagttgaagatagatttaatgcttcattag |
| | | gtacctaccatgatttgctaaaaattattaaagataaagattttttggataatgaagaaaatgaa |
| | | gatatcttagaggatattgttttaacattgaccttatttgaagatagggagatgattgaggaaa |
| | | gacttaaaacatatgctcacctctttgatgataaggtgatgaaacagcttaaacgtcgccgtt |
| | | atactggttggggacgtttgtctcgaaaattgattaatggtattagggataagcaatctggca |
| | | aaacaatattagattttttgaaatcagatggttttgccaatcgcaattttatgcagctgatccatg |
| | | atgatagtttgacatttaaagaagacattcaaaaagcacaagtgtctggacaaggcgatagt |
| | | ttacatgaacatattgcaaatttagctggtagccctgctattaaaaaaggtattttacagactgt |
| | | aaaagttgttgatgaattggtcaaagtaatggggcggcataagccagaaaatatcgttattg |
| | | aaatggcacgtgaaaatcagacaactcaaaagggccagaaaaattcgcgagagcgtatg |
| | | aaacgaatcgaagaaggtatcaaagaattaggaagtcagattcttaaagagcatcctgttga |
| | | aaatactcaattgcaaaatgaaaagctctatctctattatctccaaaatggaagagacatgtat |
| | | gtggaccaagaattagatattaatcgtttaagtgattatgatgtcgatcacattgttccacaaa |
| | | gtttccttaaagacgattcaatagacaataaggtcttaacgcgttctgataaaaatcgtggtaa |
| | | atcggataacgttccaagtgaagaagtagtcaaaaagatgaaaaactattggagacaactt |
| | | ctaaacgccaagttaatcactcaacgtaagtttgataatttaacgaaagctgaacgtggagg |
| | | tttgagtgaacttgataaagctggttttatcaaacgccaattggttgaaactcgccaaatcact |
| | | aagcatgtggcacaaattttggatagtcgcatgaatactaaatacgatgaaaatgataaactt |
| | | attcgagaggttaaagtgattaccttaaaatctaaattagtttctgacttccgaaaagatttcca |
| | | attctataaagtacgtgagattaacaattaccatcatgcccatgatgcgtatctaaatgccgtc |
| | | gttggaactgctttgattaagaaatatccaaaacttgaatcggagtttgtctatggtgattataa |
| | | agtttatgatgttcgtaaaatgattgctaagtctgagcaagaaataggcaaagcaaccgcaa |
| | | aatatttcttttactctaatatcatgaacttcttcaaaacagaaattacacttgcaaatggagag |
| | | attcgcaaacgccctctaatcgaaactaatggggaaactggagaaattgtctgggataaag |
| | | ggcgagattttgccacagtgcgcaaagtattgtccatgccccaagtcaatattgtcaagaaa |
| | | acagaagtacagacaggcggattctccaaggagtcaattttaccaaaaagaaattcggaca |
| | | agcttattgctcgtaaaaaagactgggatccaaaaaaatatggtggttttgatagtccaacgg |
| | | tagcttattcagtcctagtggttgctaaggtggaaaaagggaaatcgaagaagttaaaatcc |
| | | gttaaagagttactagggatcacaattatggaaagaagttcctttgaaaaaaatccgattgac |
| | | tttttagaagctaaaggatataaggaagttaaaaaagacttaatcattaaactacctaaatata |
| | | gtctttttgagttagaaaacggtcgtaaacggatgctggctagtgccggagaattacaaaaa |
| | | ggaaatgagctggctctgccaagcaaatatgtgaattttttatatttagctagtcattatgaaaa |
| | | gttgaagggtagtccagaagataacgaacaaaaacaattgtttgtggagcagcataagcat |
| | | tatttagatgagattattgagcaaatcagtgaattttctaagcgtgttattttagcagatgccaat |
| | | ttagataaagttcttagtgcatataacaaacatagagacaaaccaatacgtgaacaagcaga |
| | | aaatattattcatttatttacgttgacgaatcttggagctcccgctgcttttaaatattttgataca |
| | | acaattgatcgtaaacgatatacgtctacaaaagaagttttagatgccactcttatccatcaat |
| | | ccatcactggtctttatgaaacacgcattgatttgagtcagctaggaggtgacggcagcggt |
| | | gattataaagatgatgatgataaatga |
| 69 | Pseudomonas | tcagcaggacgcactgaccaggaggtacaatcaATGgataagaaatactcaataggctt |
| | cas9 with | agatatcggcacaaatagcgtcggatgggcggtgatcactgatgattataaggttccgtcta |
| | RBS | aaaagttcaaggttctgggaaatacagaccgccacagtatcaaaaaaaatcttataggggc |
| | | tcttttatttgacagtggagagacagcggaagcgactcgtctcaaacggacagctcgtaga |
| | | aggtatacacgtcggaagaatcgtatttgttatctacaggagattttttcaaatgagatggcga |
| | | aagtagatgatagtttctttcatcgacttgaagagtcttttttggtggaagaagacaagaagca |
| | | tgaacgtcatcctatttttggaaatatagtagatgaagttgcttatcatgagaaatatccaacta |
| | | tctatcatctgcgaaaaaaattggtagattctactgataaagcggatttgcgcttaatctatttg |
| | | gccttagcgcatatgattaagtttcgtggtcattttttgattgagggagatttaaatcctgataat |
| | | agtgatgtggacaaactatttatccagttggtacaaacctacaatcaattatttgaagaaaacc |
| | | ctattaacgcaagtggagtagatgctaaagcgattctttctgcacgattgagtaaatcaagac |
| | | gattagaaaatctcattgctcagctccccggtgagaagaaaaatggcttatttgggaatctca |
| | | ttgctttgtcattgggtttgacccctaattttaaatcaaattttgatttggcagaagatgctaaatt |
| | | acagctttcaaaagatacttacgatgatgatttagataatttattggcgcaaattggagatcaa |
| | | tatgctgatttgtttttggcagctaagaatttatcagatgctattttactttcagatatcctaagag |
| | | taaatactgaaataactaaggctcccctatcagcttcaatgattaaacgctacgatgaacatc |
| | | atcaagacttgactcttttaaaagctttagttcgacaacaacttccagaaaagtataaagaaat |
| | | cttttttgatcaatcaaaaaacggatatgcaggttatattgatgggggagctagccaagaag |
| | | aattttataaatttatcaaaccaattttagaaaaaatggatggtactgaggaattattggtgaaa |
| | | ctaaatcgtgaagatttgctgcgcaagcaacggacctttgacaacggctctattccccatca |
| | | aattcacttgggtgagctgcatgctattttgagaagacaagaagacttttatccatttttaaaag |
| | | acaatcgtgagaagattgaaaaaatcttgacttttcgaattccttattatgttggtccattggcg |
| | | cgtggcaatagtcgttttgcatggatgactcggaagtctgaagaaacaattaccccatggaa |
| | | ttttgaagaagttgtcgataaaggtgcttcagctcaatcatttattgaacgcatgacaaactttg |
| | | ataaaaatcttccaaatgaaaaagtactaccaaaacatagtttgctttatgagtattttacggttt |
| | | ataacgaattgacaaaggtcaaatatgttactgaaggaatgcgaaaaccagcatttctttcag |
| | | gtgaacagaagaaagccattgttgatttactcttcaaaacaaatcgaaaagtaaccgttaag |
| | | caattaaaagaagattatttcaaaaaaatagaatgttttgatagtgttgaaatttcaggagttga |
| | | agatagatttaatgcttcattaggtacctaccatgatttgctaaaaattattaaagataaagattt |
| | | tttggataatgaagaaaatgaagatatcttagaggatattgttttaacattgaccttatttgaag |
| | | atagggagatgattgaggaaagacttaaaacatatgctcacctctttgatgataaggtgatg |
| | | aaacagcttaaacgtcgccgttatactggttggggacgtttgtctcgaaaattgattaatggta |
| | | ttagggataagcaatctggcaaaacaatattagattttttgaaatcagatggttttgccaatcg |
| | | caattttatgcagctgatccatgatgatagtttgacatttaaagaagacattcaaaaagcacaa |
| | | gtgtctggacaaggcgatagtttacatgaacatattgcaaatttagctggtagccctgctatta |
| | | aaaaaggtattttacagactgtaaaagttgttgatgaattggtcaaagtaatggggcggcata |
| | | agccagaaaatatcgttattgaaatggcacgtgaaaatcagacaactcaaaagggccaga |
| | | aaaattcgcgagagcgtatgaaacgaatcgaagaaggtatcaaagaattaggaagtcaga |
| | | ttcttaaagagcatcctgttgaaaatactcaattgcaaaatgaaaagctctatctctattatctc |
| | | caaaatggaagagacatgtatgtggaccaagaattagatattaatcgtttaagtgattatgat |
| | | gtcgatcacattgttccacaaagtttccttaaagacgattcaatagacaataaggtcttaacgc |
| | | gttctgataaaaatcgtggtaaatcggataacgttccaagtgaagaagtagtcaaaaagatg |
| | | aaaaactattggagacaacttctaaacgccaagttaatcactcaacgtaagtttgataatttaa |
| | | cgaaagctgaacgtggaggtttgagtgaacttgataaagctggttttatcaaacgccaattg |
| | | gttgaaactcgccaaatcactaagcatgtggcacaaattttggatagtcgcatgaatactaaa |
| | | tacgatgaaaatgataaacttattcgagaggttaaagtgattaccttaaaatctaaattagtttc |
| | | tgacttccgaaaagatttccaattctataaagtacgtgagattaacaattaccatcatgcccat |
| | | gatgcgtatctaaatgccgtcgttggaactgctttgattaagaaatatccaaaacttgaatcg |
| | | gagtttgtctatggtgattataaagtttatgatgttcgtaaaatgattgctaagtctgagcaaga |
| | | aataggcaaagcaaccgcaaaatatttcttttactctaatatcatgaacttcttcaaaacagaa |
| | | attacacttgcaaatggagagattcgcaaacgccctctaatcgaaactaatggggaaactg |
| | | gagaaattgtctgggataaagggcgagattttgccacagtgcgcaaagtattgtccatgcc |
| | | ccaagtcaatattgtcaagaaaacagaagtacagacaggcggattctccaaggagtcaatt |
| | | ttaccaaaaagaaattcggacaagcttattgctcgtaaaaaagactgggatccaaaaaaata |
| | | tggtggttttgatagtccaacggtagcttattcagtcctagtggttgctaaggtggaaaaagg |
| | | gaaatcgaagaagttaaaatccgttaaagagttactagggatcacaattatggaaagaagtt |
| | | cctttgaaaaaaatccgattgactttttagaagctaaaggatataaggaagttaaaaaagact |
| | | taatcattaaactacctaaatatagtctttttgagttagaaaacggtcgtaaacggatgctggc |
| | | tagtgccggagaattacaaaaaggaaatgagctggctctgccaagcaaatatgtgaatttttt |
| | | atatttagctagtcattatgaaaagttgaagggtagtccagaagataacgaacaaaaacaatt |
| | | gtttgtggagcagcataagcattatttagatgagattattgagcaaatcagtgaattttctaag |
| | | cgtgttattttagcagatgccaatttagataaagttcttagtgcatataacaaacatagagaca |
| | | aaccaatacgtgaacaagcagaaaatattattcatttatttacgttgacgaatcttggagctcc |
| | | cgctgcttttaaatattttgatacaacaattgatcgtaaacgatatacgtctacaaaagaagtttt |
| | | agatgccactcttatccatcaatccatcactggtctttatgaaacacgcattgatttgagtcag |
| | | ctaggaggtgactga |
| 70 | PattKLM | ccagaatgcgtgtcagctcggcggctgtaaggtcccgcggggaacctgccacaagatcc |
| | | agaatacgcacggcgcgtctgagtgctggtacagtgtctgatatttgcgacgattgttgatct |
| | | tcagccatgcaccttccttgacacacttggcccctttggcccatagttcactctaatgattcaa |
| | | gttcaattagttgaactctaatgcgggagaggtcg |
| 71 | Ptet | tccctatcagtgatagagattgacatccctatcagtgatagagatactgagcaca |
| 72 | Pcon | ttgacagctagctcagtcctaggtataatgctagc |
| | (Pseudomonas | |
| | gRNAs) | |
| 73 | Pcon (attJ) | tttacggctagctcagtcctaggtactatgctagcta |
| 74 | att/with | tactttgcccgcagcttaggaacttgaatcattagagtgaactATGggccaaaggggcc |
| | RBS | aagtgtgtcaaggaaggtgcatggctgaagatcaacaatcgtcgcaaatatcagacactgt |
| | | accagcactcagacgcgccgtgcgtattctggatcttgtggcaggttccccgcgggacctt |
| | | acagccgccgagctgacacgcattctggatttgcccaaaagcagcgcgcatggcttgctt |
| | | gcggtgatgactgagcttgatcttctggcgcgatctgccgatggaaccctgcgtattggacc |
| | | ccactcgctcagatgggcaaatggttttctgtcgcacctcgatatagtatcgacattcaacga |
| | | ccatctcgcccagcgccacgacctcgatccctacacggtgaccctcaccgtccgcgagg |
| | | gtggcgaagtcgtttacatcggctgtcgcaactcggctcagccgcttggacacacgttccg |
| | | gatcggtatgcgtctgccggcgccatttacggccaccgggaagattctcctgtccgatctg |
| | | gggcctggtgaattgaggatgctgttctctcagtttccacagcctctgacatcaaggagtgtt |
| | | gctggcctttcgcagcttgaagaggaactggctctgacgcgcgctcgcggctactccatcg |
| | | acgatggtcagatccgcgaaggtatgctttgcattggggctgcgatacgcgattactcggg |
| | | agccgcatctgccggcattgcaatcagtctgatccgcagcgaagccagcgacgaaaaaat |
| | | cgctagccttggtgaggagcttcgcaccactgccaacgcgctttctgaaaagcttgggtac |
| | | cgatcgcagaaagactag |
| 75 | gRNA | ttgacatgaagtgttagacgtcatataatcgtggtaattctgatgccgacacttcgttggaga |
| | Enterococcus | gcaagacattgcaagttccaataaggcgtgtccgataaaagcttgagaaagcaagtcaaa |
| | array | agcctccggtcggaggcttttgactttctttacaatcagcagtcagaacttttacgaagaata |
| | | gtggtcgctcaaccttttgacactaccgagacagtgacatataataggaccttttcatagctg |
| | | gtccagttatctgagagccaaaaatggcaagttcagataaggccagaccgttaccagctta |
| | | aataagcgaaaaaaaatccttagctttcgctaaggatgatttctactatatcgtattcgtcaca |
| | | ccagattggcgtaagaagtcgctattgaaactatttgacaactgctcagcgaaatactataat |
| | | gactacggtaagagtgaagttcgcaggcttcagatccagaaatggaaagttgaagtgagg |
| | | caggtccggtagcaactcgaaagagtgaccaaaaaggggggattttatctcccctttaatttt |
| | | tccagttacacttaccctactttatcggattctgaggaacaggagactgattattgacaggtg |
| | | aacgctcagctcttataatgcctatggttggcgtcatcgtgttcaggaatagaaaacaaaagt |
| | | ttaagttattctaaggccagtccggaatcatcctaaaaaggagaagcagaaggccatcctg |
| | | acggatggcctttttgcgtttctattcacttccctcacagattcgttcagagataaaagcgttgg |
| | | taacagtttgacactggcctgacaagtccatataatgatgtcgaatcgtactaaactggtacc |
| | | attttggcgtcgaaagacgaagtaaaatgaaggcgagaccgatatcaactggaagcagtg |
| | | agcatgctgccaggtgatccccctggccacctcttttacttgtcactcagtatcagaagacg |
| | | aaaatctcaccgtgtaagttgtgttcattgacacattaggatggacgtattataatatgcccag |
| | | ctcgtgtcgtgagatgtttttttagaggaaggaattccaagttaaaaaaaggcaggaccggg |
| | | aacatgttgaaaaacagataacaaagccgggtaattcccggctttgttgtatcttttctatttac |
| | | aggcgataaggtgattgaactgacgagcaactctcctat |
| 76 | Pcon-kanR | tctcgcgcactaaacagcgactgtggaccaaaggcgaaacctcgggcaatttcctcaatgt |
| | lacZ | ggtgagcattgcgccggactgcgacaacgacacgttactggtgctggcgaatcccatcgg |
| | integration | gccaacctgccacaaaggcaccagcagctgcttcggcgacaccgctcaccagtggctgtt |
| | arm | cctgtatcaactggaacaactgctcgccgaacgtaaatctgccgacccggaaacctcctac |
| | upstream | accgccaaactgtatgccagcggctccaaacgcattgcgcagaaagtgggcgaagaag |
| | | gcgtggaaaccgcactggcagcaacggtacatgaccgctttgagctgaccaacgaggca |
| | | tctgacttgatgtatcacctgctggtgttgttgcaggatcaggatctggatttaacaacggtaa |
| | | ttgagaacctgcgtaaacggcatcagtgagtggcgtgctgagcaggtgtgatgttgttgtca |
| | | cacccggagccattg |
| 77 | Pcon-kanR | ggcaaatcgctgaatgttcgatttattcaacaaagccacgttgtgtctcaaaatctctgatgtt |
| | lacZ | acattgcacaagataaaaatatatcatcatgaacaataaaactgtctgcttacataaacagta |
| | integration | atacaaggggtgttATGagccatattcaacgggaaacgtcttgctcgaggccgcgatta |
| | | aattccaacatggatgctgatttatatgggtataaatgggctcgcgataatgtcgggcaatca |
| | | ggtgcgacaatctatcgattgtatgggaagcccgatgcgccagagttgtttctgaaacatgg |
| | | caaaggtagcgttgccaatgatgttacagatgagatggtcagactaaactggctgacggaa |
| | | tttatgcctcttccgaccatcaagcattttatccgtactcctgatgatgcatggttactcaccact |
| | | gcgatccccgggaaaacagcattccaggtattagaagaatatcctgattcaggtgaaaatat |
| | | tgttgatgcgctggcagtgttcctgcgccggttgcattcgattcctgtttgtaattgtccttttaa |
| | | cagcgatcgcgtatttcgtctcgctcaggcgcaatcacgaatgaataacggtttggttgatg |
| | | cgagtgattttgatgacgagcgtaatggctggcctgttgaacaagtctggaaagaaatgcat |
| | | aagcttttgccattctcaccggattcagtcgtcactcatggtgatttctcacttgataaccttattt |
| | | ttgacgaggggaaattaataggttgtattgatgttggacgagtcggaatcgcagaccgatac |
| | | caggatcttgccatcctatggaactgcctcggtgagttttctccttcattacagaaacggctttt |
| | | tcaaaaatatggtattgataatcctgatatgaataaattgcagtttcatttgatgctcgatgagtt |
| | | tttctaatcagaattggttaattggttgtaacactggcagagc |
| 78 | Pcon-kanR | ctacacattatctcaacacacgtaggccggataagatgcgccagcatcgcatccggcaaa |
| | lacZ | aaaaacgggcatggtgtcaccaccctgcccgtttctcttaaatgcacaataatattatttcgc |
| | integration | gttgtaattacgcagcgcgttacgcccaagcacaatccccgcgccaaccatgccgcccag |
| | arm | cagcacagccagaatcaaggtaattgctttcttcgggctatcgcgacgaataggtaacgtc |
| | downstream | ggtttcattacatagcggtaagcatgaatatcaagtttatcaacgtcaagattgtcaatatcca |
| | | gcaggttttgacgagtctgatagtagtttgatgagaacaccaacggacgggtcgcttcgtgc |
| | | ttaatcatcgactccagcgcttcgctccccaaaagaaacatagtatcctgggttacgtcctgt |
| | | gtctgttgaatctgcggctttgtcacctgcgcctgattcgcatactgcaacgcttcctgaatct |
| | | gac |

All strains of E. coli used in the study, including DH10B, MG1655, Nissle 1917, and BL21(DE3) were cultured in LB medium at 37° C. with 250 rpm shaking unless otherwise stated. Cultures derived from mouse fecal samples were also cultured in LB medium at 37° C. with 250 rpm shaking. Medium was supplemented with the following concentrations of antibiotics as necessary: 100 μg/ml ampicillin, 20 μg/ml kanamycin, and 100 μg/ml spectinomycin (Gold Biotechnology, Olivette, MO, USA). Pseudomonas strains P. putida F1, P. putida KT2440, P. stutzeri JM300, and P. syringae pv. tomato DC3000 were cultured in LB medium with 250 rpm shaking. Cultures containing exclusively P. putida F1, P. putida KT2440, or P. stutzeri JM300 were grown at 30° C. Cultures containing exclusively P. syringae pv. tomato DC3000 or mixtures containing multiple Pseudomonas strains were grown at 28° C. Medium was supplemented with the following concentrations of antibiotics as necessary: 10 μg/ml gentamycin and 50 μg/ml (P. putida F1, P. syringae pv. tomato DC3000, or P. stutzeri JM300) or 200 μg/ml (P. putida KT2440 or strain mixtures) tetracyclin (Gold Biotechnology, Olivette, MO, USA).

### gRNA Efficiency Assays

E. coli-specific gRNAs were assessed for cleavage efficiency using a chemical transformation cell death assay. Strains were first transformed with a plasmid harboring a constitutive Pₜₑₜ-cas9 expression cassette but lacking tetR. The strains were then incubated overnight in 5 mL of LB in 14 mL round bottom tubes (14-959-11B, Fisher Scientific) at 37° C. and 250 rpm. Cultures were then diluted 50× into fresh LB supplemented with the relevant antibiotic for the Cas9 plasmid in 250 mL baffled Erlenmeyer flasks. Cultures were incubated for ˜1.5 h to an OD600 of 0.4 and distributed in 1 mL aliquots in 1.7 mL centrifuge tubes (20383, GeneMate). The tubes were centrifuged at 3000×g for 2 min, the supernatant removed, and the pellets resuspended in 100 μL ice-cold 0.1 M CaCl₂. Each tube was supplemented with 10 ng of the control plasmid or a gRNA plasmid, gently mixed, and chilled on ice for 20 min. Each tube was then heat shocked in a 42° C. water bath for 60 sec and supplemented with 900 μL SOC (5 g/L yeast extract, 20 g/L tryptone, 0.5 g/L NaCl, 2.5 mM KCl, 10 mM MgCl₂, and 20 mM Glucose). The transformed cells were incubated for 60 min at 37° C. and 250 rpm. Culture dilutions were then plated on LB-agar plates with the relevant antibiotics and incubated overnight for CFU quantification.

Pseudomonas-specific gRNAs were assessed for cleavage efficiency using an electroporation cell death assay. Strains were first transformed with the pCas9-RK2K plasmid which harbors a constitutive Cas9 expression cassette. The strains were then incubated overnight in 5 mL of LB in 14 mL round bottom tubes at 28° C. (P. syringae pv. tomato DC3000) or 30° C. (P. putida F1, P. putida KT2440, or P. stutzeri JM300) and 250 rpm. Cultures were then diluted 25× into 50 mL fresh LB supplemented with the relevant antibiotic for the Cas9 plasmid in 250 mL baffled Erlenmeyer flasks. Cultures were incubated for ˜2 h to an OD600 of 0.4, centrifuged at 4000×g for 12 min, and washed three times with 50 mL of 3 mM HEPES. The pellet was resuspended in 500 μL of 3 mM HEPES and 50 μL aliquots were transferred to 1.7 mL centrifuge tubes. Each tube was supplemented with 250 ng of the control plasmid or a gRNA plasmid, gently mixed, electroporated at 2.5 kV (12358-346, Bulldog Bio; Eporator 4309, Eppendorf), and resuspended in 950 μL SOC. The transformed cells were incubated for 2.5 h at 28 or 30° C. and 250 rpm. Culture dilutions were then plated on LB-agar plates with the relevant antibiotics and incubated overnight for CFU quantification.

### E. Coli Strain-Specific Recombineering

To construct engineered E. coli variants, lambda red-mediated recombineering was utilized. The dsDNA insert was obtained by constructing a plasmid with a kanamycin-resistance cassette flanked by 500 bp arms homologous to the lacZ insertion region. The full product (both arms and insert DNA) was PCR amplified and purified by gel extraction. E. coli MG1655, DH10B, and Nissle 1917 were individually transformed with the pMP11 plasmid containing constitutive Cas9 and arabinose-inducible lambda Red expression cassettes. Individual colonies of each strain were incubated overnight in 5 mL of LB in 14 mL round bottom tubes at 30° C. and 250 rpm. Cultures were then mixed and diluted 50× in 50 mL of LB supplemented with 2% arabinose in 250 mL baffled Erlenmeyer flasks. Cultures were incubated at 30° C. and 250 rpm for an ˜2 h to an OD600 of 0.4. Cultures were chilled and washed three times in 50 mL ice cold water, resuspended in 300 μL ice cold water, and 50 μL aliquots were transferred to chilled 1.7 mL centrifuge tubes. Tubes were supplemented with 100 ng of the dsDNA insert and 100 ng of either a control plasmid or the strain-selection gRNA plasmid. The cells were electroporated at 2.5 kV, suspended in 950 μL SOC, and incubated at 30° C. and 250 rpm for 3 h. Cultures were plated on LB-agar supplemented with spectinomycin and kanamycin to select for cells that received both the control or gRNA plasmid and the integration cassette, respectively. The resulting strains were identified by colony PCR and sequencing.

### Isolating or Killing Specific Strains from Microbial Consortia

For same-genus strain mixtures, all strains were individually incubated overnight in 5 mL of LB in 14 mL round bottom tubes at 37° C. (E. coli) or 28° C. (Pseudomonas spp.) and 250 rpm. For E. coli and fecal mixtures, cultures were combined and diluted 50× into 50 mL of fresh LB in 250 mL baffled Erlenmeyer flasks and incubated for ˜1.5 h to an OD600 of 0.4. For Pseudomonas spp., cultures were combined and diluted 25× into 50 mL of fresh LB in 250 mL baffled Erlenmeyer flasks and incubated for ˜2 h to an OD600 of 0.4. The multi-strain cultures were chilled and washed three times in 50 mL ice-cold water (E. coli and fecal) or 3 mM HEPES (Pseudomonas spp.) and resuspended in 500 μL ice-cold water (E. coli and fecal) or 3 mM HEPES (Pseudomonas spp.) and 50 μL aliquots transferred to chilled 1.7 mL centrifuge tubes. The multi-strain cells were then transformed with 10 ng (E. coli) or 250 ng (Pseudomonas spp.) of the control plasmid or relevant test plasmid harboring cas9 and strain-specific gRNA cassettes and resuspended in 950 μL SOC. After 60 min (E. coli) or 2.5 h (Pseudomonas spp.), the transformations were plated for the specified cell quantification method.

For NGS strain quantification, transformations were plated onto LB-agar plates supplemented with spectinomycin (E. coli) or gentamycin (Pseudomonas) and incubated overnight at the respective temperature. All colonies were mixed together and resuspended in 5 mL of LB. The resuspension was then used as a template for a mixed colony PCR with primers harboring NGS adapter sequences (Table 6). PCR products were gel purified and submitted to Genewiz for Amplicon-EZ sequencing. For antibiotic-based quantification, transformations were serially diluted and each dilution was plated onto four LB-agar plates with antibiotics matching the resistances of the four strains.

TABLE 6
Strain identification sequences

| SEQ ID NO: | Part | Nucleotide Sequence |
|---|---|---|
| 79 | E. coli identification primer with | acactctttccctacacgacgctcttccgatctg |
| | NGS adapter Forward | gtgatgtgtactgactgaatgg |
| 80 | E. coli identification primer with | gactggagttcagacgtgtgctcttccgatctg |
| | NGS adapter Reverse | catttgatcgcaacagcacg |
| 81 | E. coli DH10B identification | ggtgatgtgtactgactgaatggcgctggcct |
| | sequence | caccgcccggaaccggaacttcggtgccaat |
| | | gacatagctcagttgctcacgctggcaatctgt |
| | | cgccacactttccgcagcaaagcaaagcaca |
| | | gcagctcgttccgcaaccgtttctggtgctaac |
| | | ggtatgggatcccccgcgcaggacattgacg |
| | | catcaagatgaattttactgaagccggcacga |
| | | acatattcctataccagctcgacggatttttcca |
| | | tcgccgcatccgcattttcttgctgccagcagtt |
| | | tggccccagatgatcgccgccgagaataatg |
| | | cgttcgcgtgcaaacccaactttatcggcaatc |
| | | gtaaaaacaaattcgcgaaagtctgccggtgt |
| | | cattccggtataaccgccaaattgattgacctg |
| | | gtttgacgttgcttcaatcagcactttgcgcgtg |
| | | ctgttgcgatcaaatgc |
| 82 | E. coli MG1655 identification | ggtgatgtgtactgactgaatggcgctggcct |
| | sequence | caccgcccggaaccggaacttcggtgccaat |
| | | gacatagctcagttgctcacgctggcaatctgt |
| | | cgccacactttccgcagcaaagcaaagcaca |
| | | gcagctcgttccgcaaccgtttctggtgctaac |
| | | ggtatgggatcccccgcgcaggacattgacg |
| | | catcaagatgaattttactgaagccggcacga |
| | | acatattcctttaccagctcgacggatttttccat |
| | | cgccgcatccgcattttcttgctgccagcagttt |
| | | ggccccagatgatcgccgccgagaataatgc |
| | | gttcgcgtgcaaacccaactttatcggcaatcg |
| | | taaaaacaaattcgcgaaagtctgccggtgtc |
| | | attccggtataaccgccaaattgattgacctgg |
| | | tttgacgttgcttcaatcagcactttgcgcgtgc |
| | | tgttgcgatcaaatgc |
| 83 | E. coli Nissle identification | ggtgatgtgtactgactgaatggcgctggcct |
| | sequence | caccgcccggaaccggaacttcggtgccaat |
| | | gacatagttcagttgctcacgctggcaatcagt |
| | | cgccacactttccgccgccagacaaagcaca |
| | | gcggcacgttcagcaaccgtttctggcgctaa |
| | | cggtatggaatcgtcagcgcaggacattgac |
| | | gcatcaagatgaattttactgaagccggcacg |
| | | aacatatgcctttaccagctcgacggatttttcc |
| | | atcgccgcatccgcattttcttgctgccagcag |
| | | tttggtcccagatggtcgccgccgagaataat |
| | | acgctcacgagcaaatccgactttatcggcaa |
| | | tcgcaaaaacaaattcgcgaaagtctgccggt |
| | | gtcattccggtataaccgccaaattgattgacc |
| | | tggtttgacgttgcttcaatcagcactttacgcg |
| | | tgctgttgcgatcaaatgc |
| 84 | E. coli BL21(DE3) identification | ggtgatgtgtactgactgaatggcgctggcct |
| | sequence | caccgcccggaaccggaacttcggtaccaat |
| | | gacatagctcagttgctcacgctggcaatctgt |
| | | coccacactttccgcagcaaagcaaagcaca |
| | | gcagctcgttccgcaaccgtttctggtgctaac |
| | | ggtatgggatcccccgcgcaggacattgacg |
| | | catcaagatgaattttactgaagccagcacga |
| | | acatatgcttttaccagctcgacggatttttccat |
| | | cgccgcatccgcattttcttgctgccagcagttt |
| | | ggccccagatgatcgccgccgagaataatgc |
| | | gttcgcgtgcaaacccaactttatcggcaatcg |
| | | taaaaacaaattcgcgaaagtctgccggtgtc |
| | | attccggtataaccgccaaattgattgacctgg |
| | | tttgacgttgcttcaatcagcactttgcgcgtgc |
| | | tgttgcgatcaaatgc |
| 85 | Pseudomonas identification primer | acactctttccctacacgacgctcttccgatctc |
| | with NGS adapter Forward | ttgtagttgctgtagaac |
| 86 | Pseudomonas identification primer | gactggagttcagacgtgtgctcttccgatcta |
| | with NGS adapter Reverse | tggccagcaactcgctg |
| 87 | P. putida F1 identification sequence | cttgtagttgctgtagaactcgtcgatttcatcc |
| | | agcagcaggcgaatgcggtgctgggcccgc |
| | | tgcaggcgcttgctggtgcgcacgatgccga |
| | | cgtagtcccacatgaagcgccgcagttcgtcc |
| | | cagttgtgcgcaatgatcacgtcctcgtccga |
| | | gtcggtcacctggctggcgtcccagccgggc |
| | | agggccttgggcatgtccacttgctccaggtg |
| | | cgcctggatgtcggcagcggcggcgcgacc |
| | | gtacacgaaacattccaacagcgagttgctgg |
| | | ccat |
| 88 | P. putida KT2440 identification | cttgtagttgctgtagaactcgtcgatttcatcc |
| | sequence | agcagcaggcgaatgcggtgctgggcccgc |
| | | tgcaggcgcttgctggtgcgcacgatgccga |
| | | cgtagtcccacatgaagcgccgcagttcgtcc |
| | | cagttgtgcgcaatgatcacgtcctcgtccga |
| | | gtcggtcacctggctggcgtcccagccgggc |
| | | aaggccttgggcatggccacttgctccaggtg |
| | | cgcctggatgtcggcagcggcggcgcgacc |
| | | gtacacaaaacattccagcagcgagttgctgg |
| | | ccat |
| 89 | P. stutzeri JM300 identification | cttgtagttgctgtagaactcgtcgatttcgtcc |
| | sequence | aacagcaggcggactcggtgctgggcgcgc |
| | | gtcaggcgcttgtcggtgcggacgatgccca |
| | | cgtagtcccacatgaaccgccgcagctcgtcc |
| | | cagttgtgagcgatgatcacgtcttcatccgag |
| | | ttggtgacctggctggcgtcccattgcggaag |
| | | gttggtcggtccgtcgatttccggtagctcgcg |
| | | caggatgtcgcgtgcggcggaacgtgcatag |
| | | acgaagcactcgagcagcgagttgctggccat |
| 90 | P. syringae pv. tomato DC3000 | cttgtagttgctgtagaactcatcaatttcgtcg |
| | identification sequence | agcaacaaccgcacacggtgctgggcgcgc |
| | | tgcaagcgtttgttggtgcgcacgataccgac |
| | | gtaatcccacatgaacctccttaattcgtccca |
| | | gttgtgggcgatgatcacgtcttcgtccgagtc |
| | | ggttacctggctggcatcccacgctggcaggt |
| | | cagtcggcatcggcacactggacaactgcttt |
| | | tcgatgtctgcggctgccgagcgggcgtaga |
| | | cgaagcattccagcagcgagttgctggccat |

For multi-genus strain mixtures, each strain was individually incubated overnight in 5 mL of LB in 50 mL glass culture tubes (47729-586, VWR International) at 30° C. and 250 rpm. Cultures were combined at an OD600 ratio of 1:1:1:1, diluted 50× into fresh LB, and incubated for 2 h. Cultures were then chilled, washed three times with 50 mL ice-cold water, resuspended in 500 μL water, aliquoted at 50 μL, and transformed with 100 ng of the control plasmid or test plasmid. Transformations were resuspended in 950 μL SOC, incubated for 2.5 h at 30° C. and 250 rpm, and plated for qPCR-based strain quantification. After 24 h of incubation at 30° C., all resulting colonies were combined, and the genomic DNA was extracted using the ZR Fungal/Bacterial DNA MidiPrep kit (D6105, Zymo Research). The genomic DNA was used as the template for quantitative PCR (qPCR) reactions using qPCR primers for each strain (Table 7). qPCR primer pairs for each strain were designed following previously described guidelines.

TABLE 7
qPCR primers

| | SEQ | | SEQ | |
|---|---|---|---|---|
| Strain | ID NO: | Primer 1 | ID NO: | Primer 2 |
| E. coli Nissle | 91 | TCGTTATAGCA | 92 | TTCACAGCGAT |
| 1917 | | AACCACGGC | | TGGGTGCTG |
| P. putida F1 | 93 | TCAAGGTCTAC | 94 | CGAAATTGGCG |
| | | CAGCGCAAC | | ATCGACTGG |
| S. typhimurium | 95 | TGTACCACCCC | 96 | TTGCCGCCCGC |
| | | ATCACATCC | | TGAATAATC |
| R. opacus | 97 | GAAAGCGTTCG | 98 | CCAGGTCAAAC |
| PD630 | | AGCACGTCG | | ATATTCTCGGA |
| | | | | GC |

SsoAdvanced Universal SYBR Green Supermix (1725270, BioRad), Simi-Skirted 96-well PCR Plates (T-3070-1, GeneMate), and the standard suggested CFX Connect Real-Time System (Bio-Rad) protocols were used for the qPCR reactions. The 2^{-ΔΔCT} analysis method was then used to quantify relative population values across samples.

### Liposome Synthesis, Packaging, and Killing Assays

Liposomes were generated as previously described. The neutral lipid 1,2-dioleoyl-sn-glycero-3-phosphoethanolamine (DOPE; 76548, Millipore Sigma) and cationic lipid N-[1-(2,3-dioleoyloxy)propyl]-N,N,N-trimethylammonium chloride (DOTAP; D6182, Millipore Sigma) were individually dissolved in chloroform at a concentration of 5 mM. The two lipids were then mixed at a 1:1 molar ratio in a 250 mL Buchner flask. The chloroform was removed under a vacuum overnight. The lipid film was rehydrated in 20 mM HEPES at a final concentration of 5 mM of each lipid. The mixture was vortexed for 1 min and sonicated in a 40° C. bath sonicator (Branson M3800H) for 30 min. Half of the mixture was removed after 5 min of sonication for protocol optimization experiments. Liposomes were stored at 4° C. until used. To package the liposomes with plasmid DNA, liposomes were diluted to the specified concentration in 1 mL of 20 mM HEPES and mixed with 1 μg of the plasmid. The mixture was then subjected to five 1-2 min freeze-thaw cycles between liquid nitrogen and a 40° C. water bath.

To assess the antimicrobial activity of the DNA-loaded liposomes, E. coli MG1655, DH10B, BL21(DE3), and Nissle 1917, each harboring a plasmid with a different antibiotic resistance gene, were individually incubated overnight in 5 mL of LB in 14 mL round bottom tubes at 37° C. and 250 rpm. Cultures were combined and diluted 40× into 40 mL of fresh LB in 250 mL baffled Erlenmeyer flasks and incubated an additional ˜2 h to an OD600 of ˜0.6. 0.5 mL of the exponential phase cultures were aliquoted into 1.7 mL centrifuge tubes and centrifuged at 3000×g for 2 min. The supernatant was then removed, and the pellet was washed with 1 mL 20 mM HEPES. The tube was again centrifuged at 3000×g for 2 min and the supernatant was removed. The pellet was then resuspended in 0.5 mL of the DNA-loaded liposome mixture. The mixture of liposome and E. coli was incubated at 37° C. and 250 rpm for 30 min. The centrifuge tubes were supplemented with 0.5 mL of SOC medium and returned to the incubator for an additional 60 min. For CFU quantification and cell type identification, cultures were plated onto four LB-agar plates, each supplemented with a different antibiotic.

### Analysis of Next-Generation Sequencing

Amplicon-EZ next-generation sequencing was performed by Genewiz to sequence individual DNA strands from purified colony PCR samples obtained from pooled cell samples. The resulting Fastq.gz files were analyzed using custom Python scripts. Two Fastq.gz files were obtained for each sequencing sample (one forward and one reverse), however, only forward reads were analyzed to avoid double counting. Individual sequencing reads were extracted from the files and assessed for read length and sequence. Only sequences of at least 240 nucleotides long were considered. Sequences were compared to the wildtype sequences and counted for the relevant strains: E. coli Nissle 1917, MG1655, DH10B, and BL21(DE3) or P. putida F1, putida KT2440, stutzeri JM300, and syringae DC3000 (Supplementary Table 6). Only sequencing reads with a perfect match to one of the strains of interest were counted.

### Quantification of the Frequency of gRNA Target Sequences in Random DNA

The probability that a gRNA target sequence, including the PAM sequence, will appear in a randomly generated nucleotide sequence was calculated using Equation 1. The equation inaccurately assumes that every nucleotide in the sequence is independently generated without bias. This results in an overestimation of the probability of random occurrence relative to the occurrences observed in practice when multiple sequence-similar strains are considered.

P=1−(1−(0.25)^{PAM+gT})^{N-PAM-gT} (1)

where P=Probability that the gRNA target sequence is present in a random nucleotide sequence; PAM=Number of non-random nucleotides in the PAM sequence; gT=Number of nucleotides in the gRNA target site being considered for specificity; and N=Length of the random nucleotide sequence.

### Quantification and Statistical Analysis

All statistical tests were performed using GraphPad Prism or Excel. All statistical details of experiments, including the definitions of center, significance criteria, and sample size can be found in the figure legends. Sample sizes were chosen based on our previous work and the literature, and represent sample sizes routinely used for these methods. No sample size calculations were performed during the design of experiments. Samples were randomized during group assignment in all experiments. No samples were excluded from analyses. The Investigators were not blinded to allocation during experiments and outcome assessment.

What is claimed is:

1. A computer-implemented method of producing at least one gRNA sequence for use in CRISPR-Cas gene editing of microbial organisms, each gRNA sequence comprising a protospacer adjacent motif (PAM) sequence and a target nucleotide sequence, the method comprising:
receiving, at a computing device, at least one non-target strain genome sequence, at least one target strain genome sequence, the PAM sequence, a PAM orientation, a specificity threshold, and a target length;
identifying, using the computing device, a plurality of candidate gRNA sequences within the at least one target strain genome sequence, based on the PAM nucleotide sequence, the PAM orientation, and the target length;
selecting, using the computing device, at least one broad-specificity gRNA sequence from the plurality of candidate gRNA sequences, wherein each broad-specificity gRNA sequence is contained within all of the at least one target strain genome sequences;
identifying, using the computing device, a plurality of non-target gRNA sequences within the at least one non-target strain genome sequence, based on the PAM nucleotide sequence, the PAM orientation, and the target length; and
selecting, using the computing device, at least one strain-specific gRNA sequence from the at least one broad-specificity gRNA sequence based on the specificity threshold, wherein the at least one strain-specific gRNA sequence is not contained within any of the non-target strain gRNA sequences.

2. The method of claim 1, wherein the candidate gRNA sequence, broad-specificity gRNA sequence, non-target gRNA sequence, non-target gRNA sequence, and strain-specific gRNA sequence each comprise the PAM nucleotide sequence and a target nucleotide sequence comprising the target length of nucleotides, the PAM nucleotide sequence and the target sequence arranged according to the PAM orientation selected from 5-(PAM nucleotide sequence)-(target nucleotide sequence)-3′ or 5′-(target nucleotide sequence)-PAM nucleotide sequence)-3′.

3. The method of claim 2, wherein the specificity threshold comprises a minimum number of nucleotide mismatches between the target sequence of each strain-specific gRNA sequence and the target sequence of all non-target strain gRNA sequences.

4. The method of claim 3, wherein the specificity threshold ranges from 0 nucleotides (nt) to about 4 nt.

5. The method of claim 4, wherein the specificity threshold is at least 3 nt.

6. The method of claim 2, wherein the target length ranges from about 10 nt to about 20 nt.

7. The method of claim 6, wherein the target length is 20 nt.

8. The method of claim 3, wherein selecting the at least one strain-specific gRNA sequence from the at least one broad-specificity gRNA sequence further comprises:
generating, using the computing device, nucleotide sequence permutations within each target region of each non-target gRNAS sequence ranging from at least one nucleotide up to the specificity threshold number of nucleotides relative to the at least one non-target gRNA sequence; and
comparing, using the computing device, each target sequence of each broad-specificity gRNA sequence to the plurality of nucleotide sequence permutations and discarding each broad-specificity gRNA sequence that matches any of the nucleotide sequence permutations, and selecting the remaining broad-specificity gRNA sequences as the strain-specific gRNA sequences.
